(19) United States
(12) Patent Application Publication
(10) Pub. No.: US 2016/0184321 A1
(43) Pub. Date: Jun. 30, 2016

(54) COMPOSITIONS AND THEIR USE IN ORAL DOSING REGIMENS

(71) Applicant: Lipocine Inc., Salt Lake City, UT (US)

(72) Inventors: Mahesh Patel, Salt Lake City, UT (US); Nachiappan Chidambaram, Sandy, UT (US); Satish Nachaegari, Salt Lake City, UT (US); Srinivasan Venkateshwaran, Salt Lake City, UT (US)

(73) Assignee: Lipocine Inc., Salt Lake City, UT (US)

(21) Appl. No.: 14/864,783

(22) Filed: Sep. 24, 2015

Related U.S. Application Data
(60) Provisional application No. 62/054,927, filed on Sep. 24, 2014.

(51) Int. Cl. A61K 31/568

(52) U.S. Cl. CPC A61K 31/568

(57) ABSTRACT

Described herein are oral pharmaceutical compositions and their use in testosterone replacement therapy applications.

### CROSS-REFERENCE TO RELATED APPLICATIONS

This application claims the benefit of U.S. Provisional Application No. 62/054,927 filed Sep. 24, 2014 which is incorporated herein by reference in its entirety.

### FIELD OF THE INVENTION

This disclosure relates to compositions having testosterone undecanoate for oral administration to subjects and their use in dosing regimens.

### BACKGROUND

Testosterone replacement therapy (“TRT”) products on the U.S. market (e.g., approved by the U.S. Food & Drug Administration) include gels applied to the skin, transdermal patches applied to the skin, and injectables. The dosing schemes for these products are highly variable indicating that dose titration with testosterone products is not a simple matter. Determining dose titration for a completely new class of product (oral testosterone undecanoate) never previously approved in the United States by the FDA is expected to be even more difficult. Described in more detail below are the dose titration recommendations for a number of approved testosterone replacement products obtained from their respective labels.

The AndroGel® label indicates that the starting dose is 40.50 mg per day which can be up-titrated to 81 mg per day or down to 20.25 mg. Thus, the range is 50% to 200% of the starting dose and there are four recommended daily doses. Furthermore, the dose titration should be based on pre-morning dose testosterone levels. See page 4 of the AndroGel® 1.62% label.

DEPO®-Testosterone is recommended at 50 mg to 400 mg every 2-4 weeks with dose adjustments based on patients response and adverse reactions.

ANDRODERM® is recommended at 4 mg, patch applied at night on a daily basis. Titration is based on serum testosterone levels during the early morning with a maximum of 6 mg daily or a minimum of 2 mg daily. Thus, there are 3 doses recommended with the range being 50% or 150% of the starting doses.

Testim® is recommended at 50 mg per day (preferably in the morning) with morning serum testosterone being measured approximately 14 days after start of therapy—if the serum level is below the normal range or clinical response is not achieved then the dose should be increased to 100 mg per day. Thus, the only option for this product is an increase to 200% of the starting dose.

Outside of the US, there is an approved oral TRT product on the market referred to as Andriol® or Andriol® Testocaps (or other brand names). The Andriol® label (Canada) recommends a starting testosterone undecanoate dose of 120-160 mg total daily dose followed by a maintenance dose of 40-120 mg. It is recommended that the dose should be adjusted according to the response of the individual patient.

As can be seen, there is little rhyme or reason to the dose titration protocols amongst these testosterone products with the dose titrations ranging from 50% to 200% of the initial dose and typical titration steps of 50%. The timing of the determination of serum testosterone compared to last dose is highly variable and is not even specifically recommended for some products. For example, AndroGel® serum T is measured morning pre-dose (e.g., almost 24 hours after previous dose); ANDRODERM® serum T is measured early morning after evening dose (e.g., if applied at 10 PM and early morning is 8 hours this would be 10 hours post dosing). All of these products (except DEPO®-testosterone) are transdermal in nature e.g., the same basic route of administration—across the skin.

There is a need for TRT products having simple, robust dosing regimens and titrations.

### SUMMARY

Disclosed herein is an oral testosterone replacement therapy (“TRT”). The TRT described herein provides for three different daily doses of testosterone undecanoate formulated for oral administration that are typically divided into two administrations e.g., a morning dose and an evening dose. Typically, the doses of testosterone undecanoate are administered with a meal. The three daily doses of testosterone undecanoate are in the 280-320 mg range, the 430 mg to 470 mg range, and the 580 mg to 620 mg range. These daily doses are provided by two to eight unit dosage forms per day. According to this TRT, a subject or patient e.g., hypogonadal male starts on an initial dose or initial dosing regimen that provides a specific amount of testosterone per day for an initial period of time (e.g., greater than one, two, or three weeks). After the initial period of time, a dose titration measurement is made. The dose titration measurement is made by determining serum testosterone levels at a specific time after a single dose of the initial regimen. Depending on the serum testosterone level obtained from the dose titration measurement, the patient can receive a maintenance regimen that has the same daily dose as the initial regimen or the daily dose is increased or decreased. Typically, the patient or subject is then maintained on the maintenance regimen, although one or more additional dose titration measurements or dose titrations can be made.

It was unexpectedly found that this TRT provides numerous benefits to individuals or populations of individuals in need of such therapy. As described herein, the majority of patients or subjects using this therapy have a daily dose that is that of the initial regimen. Moreover, the majority of patients or subjects using this therapy have either no dose titrations or one dose titration (e.g., change of daily dose). Additionally, a large percentage of subjects e.g., greater than 35% require no dose titration. The TRT described herein provides serum testosterone C_{avg} values within the range of 300 to 1140 ng/dL in greater than 75% of the subjects including greater than or equal to 80% or 85%.

### BRIEF DESCRIPTION OF THE DRAWINGS

FIG. 1 shows a diagram of a clinical trial design for TRT.

FIG. 2 shows a pharmacokinetic profile for 53 patients receiving TRT as described herein at weeks 3, 7, and 13 as indicated.

FIG. 3 shows pharmacokinetic profiles at week 3 and week 13 as indicated.

### DETAILED DESCRIPTION

As described herein an oral TRT is provided. The TRT has three different daily doses which commence with an initial dosing regimen having a specific daily dose of testosterone undecanoate that last for a period of time e.g., at least 1, 2, 3, 4, 5, 6, 7, 8, 9, 10, 11, 12, 13, 14, 15, or 16 weeks. After this period of time on the initial dosing regimen, a dose titration measurement or assessment is made. The purpose of the dose titration assessment or measurement is to determine if the daily dose should remain the same as the daily dose of the initial regimen or whether the daily dose should be increased or decreased. The dose titration measurement or assessment is made by determining serum testosterone concentrations within a specific amount of time (e.g., window of time) after administration of a single dose of the initial regimen at steady state. Three options are possible based on the result of this measurement or assessment. A level of serum testosterone that is too high will result in a decrease in the total daily dose of testosterone undecanoate, a level of serum testosterone that is too low will result in an increase in the daily dose of testosterone undecanoate and intermediate levels of serum testosterone will result in no change of the daily dose of testosterone undecanoate. Due to the innovative clinical trial design, the inventors have determined a dose titration scheme that is particularly robust and beneficial to patients receiving this therapy. In part, this was made possible by the determination of detailed pharmacokinetics over a 24 hour period (e.g., determination of serum testosterone levels often (e.g., every hour or two hours or so over a 24 hour period) while receiving a particular daily dose of testosterone undecanoate) and a correlation of this data to a single time point (e.g., within a certain time window) determination.

It was surprisingly found that the TRT described herein provides numerous benefits to individuals or populations of individuals in need of such therapy. The clinical trial described in the examples and figures show that the majority of patients or subjects using this therapy have a daily dose that is the same as the daily dose of the initial regimen. This indicates that the TRT initial dosing regimen daily dose is adequate for providing therapeutic levels of testosterone to a substantial portion of patients. Moreover, the majority of patients or subjects using this therapy have either no dose titrations or one dose titration (e.g., change of daily dose). Additionally, a large percentage of subjects e.g., greater than 35% require no dose titration. The TRT described herein provides C_{avg} serum testosterone values within the range of 300 to 1140 ng/dL in greater than 75% of the subjects, including greater than or equal to 80% or 85%. The TRT described herein provides for three different daily doses of testosterone undecanoate formulated for oral administration that are typically divided into two administrations e.g., a morning dose and an evening dose. The three daily doses of testosterone undecanoate are in the 280-320 mg range, 430 mg to 470 mg range, and 580 mg to 620 mg range. These daily doses are provided by two to eight unit dosage forms per day.

In one embodiment, according to this TRT, a subject or patient e.g., hypogonadal male starts on an initial dose or initial dosing regimen that provides a specific amount of testosterone undecanoate per day for an initial period of time (e.g., greater than one, two, or three weeks). This initial daily dose is in the range of about 430 mg to 470 mg TU per day (or 435 mg to 465 mg TU per day, 440 mg to 460 mg TU per day, 445 mg to 455 mg TU per day, or about 450 mg per day). After the initial period of time, a dose titration measurement or assessment is made. The dose titration measurement is made by determining serum testosterone levels at a specific time (e.g. within 1 to 12 hours, 1 to 11 hours, 1 to 10 hours, 1 to 9 hours, 1 to 8 hours, 1 to 7 hours, 1 to 6 hours, 1 to 5 hours, 1 to 4 hours, 2 to 10 hours, 2 to 9 hours, 2 to 8 hours, 2 to 7 hours, 2 to 6 hours, 2 to 5 hours, 2 to 4 hours, 1 to 3 hours, 2 to 3 hours, 3 to 4 hours, 4 to 5 hours, 3 to 5 hours, 4 to 6 hours, 3 to 6 hours, 3 to 8 hours, or 4 to 6 hours; or at about 1, 2, 3, 4, 5, 6, 7, 8, 9, 10, 11, or 12 hours±0.5, 1, 1.5, or 2 hours) after a single dose of the initial regimen when the patient is at steady state. Depending on the serum testosterone level obtained from the dose titration measurement, the patient can receive a maintenance regimen that has the same daily dose as the initial regimen or the daily dose is increased or decreased. Typically, the patient or subject is then maintained on the maintenance regimen, although one or more additional dose titration measurements or dose titration can be made. The therapy described herein is typically administered as a twice a day therapy with a meal, so a 300 mg TU dose is administered as 150 mg with a meal twice a day; a 450 mg dose is administered as 225 mg with a meal twice a day; and a 600 mg dose is administered as 300 mg with a meal twice a day.

In another embodiment, according to this TRT, a subject or patient e.g., hypogonadal male starts on an initial dose or initial dosing regimen that provides a specific amount of testosterone undecanoate per day for an initial period of time (e.g., greater than one, two, or three weeks). In one aspect, the initial daily dose of TU is in the range of about 445 mg to 455 mg TU per day, or about 450 mg per day (e.g., about 222.5 mg to 227.5 mg or about 225 mg BID (e.g., once in the morning and once in the evening). After the initial period of time, a dose titration measurement or assessment is made. The dose titration measurement is made by determining serum testosterone levels at a specific time (e.g., about 3 to 6 hours or at about 3-4 hours, 4-5 hours, or 5-6 hours) after administration of a single dose of the initial regimen with a meal when the patient is at steady state. Depending on the serum testosterone level obtained from the dose titration measurement, the patient can receive a maintenance regimen that has the same daily dose as the initial regimen or the daily dose is increased or decreased. In one example, if the subject's serum testosterone level 3-6 hours after single dose administration is less than 300 ng/dL, 250 ng/dL, 200 ng/dL, 175 ng/dL, 150 ng/dL, 125 ng/dL or 100 ng/dL, the dose of TU is adjusted up (e.g., an additional 75 mg TU per dose or 150 mg TU per day). In another example, if the subject's serum testosterone level 3-6 hours after single dose administration is greater than 1000 ng/dL, 1050 ng/dL, 1100 ng/dL, 1150 ng/dL, 1200 ng/dL or 1250 ng/dL, the dose of TU is adjusted down (e.g., decreased by 75 mg TU per dose or 150 mg TU per day). In another example, if the subject's serum testosterone level 3-6 hours after single dose administration is between 1300 ng/dL and 100 ng/dL, 1250 ng/dL and 110 ng/dL, or 1200 ng/dL and 125 ng/dL, the dose of TU remains the same. In a more specific example, if the subject's serum testosterone level 3-6 hours after single dose administration is less than about 125 ng/dL, the dose of TU is adjusted up (e.g., an additional 75 mg TU per dose or 150 mg TU per day). In another example, if the subject's serum testosterone level 3-6 hours after single dose administration is greater than about 1200 ng/dL, the dose of TU is adjusted down (e.g., decreased by 75 mg TU per dose or 150 mg TU per day); and if the subject's serum testosterone is between about 125 ng/dL and 1200 ng/dL, the dose of TU remains the same. Typically, the patient or subject is then maintained on the maintenance regimen, although one or more additional dose titration measurements or dose titrations can be made. The therapy described herein is typically administered as a twice a day therapy with a meal, so a 300 mg TU dose is administered as 150 mg with a meal twice a day; a 450 mg dose is administered as 225 mg with a meal twice a day; and a 600 mg dose is administered as 300 mg with a meal twice a day. In some aspects of this embodiment, the serum testosterone level is determined at least 7, 8, 9, 10, 11, 12, 13, 14, 15, 16, 17, 18, 19, 20, or 21 days after starting on the TRT or after starting on a new daily dose of the TRT. In a more specific aspect, the serum testosterone level is determined at least 10, 11, 12, 13, 14, 15 or 16 days after starting on the TRT or after starting on a new daily dose of the TRT. In a more specific aspect, the serum testosterone level is determined at least 14 days after starting on the TRT or after starting on a new daily dose of the TRT.

Table A below shows a titration or dose adjustment scheme determined from the study described in the Examples. Specifically, 24 hour pharmacokinetic profiles for serum testosterone were determined for hypogonadal subjects receiving 225 mg TU twice daily with a meal after 3 weeks of treatment. The titration or dose adjustment was made based on the serum testosterone C_{avg0-24} value calculated from the 24 hour pharmacokinetic profile (or the Cₘₐₓ value). Another titration or dose adjustment was made similarly at 7 weeks. The titration or adjustment criteria used during the study is shown in the first column of Table A with the titration decision (dose adjustment) is shown in column 3. The middle column shows the titration metric that corresponds at 3-6 hours after single dose administration of TU with a meal at steady state to what was determined using the 24 hour pharmacokinetic profile methodology.

TABLE A

| Cavg0-24* | Titration Metric | |
|---|---|---|
| From 24 Hour PK | 3-6 Hours after | |
| Study at 3 & 7 weeks | Single Dose | Titration Decision |
| Lower than 300 | Less than 125 ng/dL | Up 75 mg per dose |
| ng/dL | | or 150 mg per day |
| 300-1140 ng/dL | 125-1200 ng/dL | No change |
| Greater than 1140 | Greater than 1200 ng/dL | Down 75 mg per dose |
| ng/dL | | or 150 mg per day |

*Cmax greater than 1500 ng/dL were down titrated regardless of C_{avg0-24}

At the end of titration phase in the Study described in the Examples, about 52% subjects ended up at 25 mg testosterone undecanoate BID dose (starting dose), about 32% and 16% ended up at 150 mg testosterone undecanoate BID and 300 mg testosterone undecanoate BID dose, respectively. Overall, about 41% subjects never required any titration and about 48% subjects underwent one titration. This result is surprising in view of the active control arm (topical gel) of this study, which at the end of titration phase about 61% subjects required two titrations.

In one specific embodiment, the TRT dose adjustment schema is as follows: If the measured serum testosterone concentration is below about 125 ng/dL about 3-6 hours after morning single dose administration of TU (e.g., within the range of about 140 mg to 240 mg TU per dose (or 280 mg to 480 mg per day)) with a meal at steady state, the dose of TU is increased. in a specific aspect of this embodiment, the single dose is about 140, 145, 150, 155, or 160 mg of TU. In a specific aspect of this embodiment, the single dose is about 215, 220, 225, 230, or 235 mg of TU. In a specific aspect of this embodiment, the single dose is about 300 mg of TU and an additional assessment is considered if the serum testosterone is below about 125 ng/dL. In a specific aspect, the dose is increased by 75 mg as shown below in Table B.

TABLE B

| Current dose | Dose changed to |
|---|---|
| 225 mg BID | 300 mg BID |
| 150 mg BID | 225 mg BID |
| 300 mg BID | Consider additional assessment |

In one specific embodiment, the TRT dose adjustment schema is as follows: If the serum testosterone concentration exceeds 1200 ng/dL about 3-6 hours after morning single dose administration e.g., within the range of about 215 mg to 320 mg TU per dose (or 430 mg to 620 mg per day)) with a meal at steady state, the dose of TU is decreased. In a specific aspect of this embodiment, the single dose is about 215, 220, 225, 230, or 235 mg of TU. In a specific aspect of this embodiment, the single dose is about 290, 295, 400, 305, or 310 mg of TU. In a specific aspect of this embodiment, the single dose is about 150 mg of TU and an additional assessment is considered if the serum testosterone is above about 1200 ng/dL. in a specific aspect of this embodiment, the dose of TU is decreased by 75 mg as shown below in Table C.

TABLE C

| Current dose | Dose changed to |
|---|---|
| 225 mg BID | 150 mg BID |
| 150 mg BID | Consider additional assessment |
| 300 mg BID | 225 mg BID |

In one specific embodiment, the TRT dose adjustment schema is as follows: If the serum testosterone concentration is between about 125 ng/dL and 1200 ng/dL at about 3-6 hours after morning single dose administration (e.g., one of about 140 mg, 145 mg, 150 mg, 155 mg, 160 mg, 215 mg, 220 mg, 225 mg, 230 mg, 235 mg, 290 mg, 295 mg, 300 mg, 305 mg, or 310 mg TU) with a meal at steady state, no dose change is needed.

The dose titration of the TRT described herein was found to be surprisingly robust and beneficial for patients receiving the therapy. In one aspect, the dose titration is made or determined by determining a detailed pharmacokinetic profile for a set of patients (e.g., at least 10, 20, 30, 40, 50, 60, 70, 80, 90 or 100) receiving the therapy for a period of time (e.g., 24 hours). The detailed 24 hour pharmacokinetic profile yields a C_{avg} value (0-24). Patients having a C_{avg} for serum testosterone of greater than 1140 ng/dL are down titrated, a C_{avg} of less than 300 ng/dL are up titrated and a C_{avg} in the range of 300-1140 ng/dL are maintained at the same dose. In one aspect, if Cₘₐₓ>1500 ng/dL, then the dose is down titrated irrespective of C_{avg} value. In practice, the detailed pharmacokinetic profile is used to determine a time point or window that is predictive of the C_{avg} values or correlated thereto. Thus, the determination of serum testosterone at the predictive or correlative time point or window can be used to determine the dose titration. In a specific aspect, the up or down titration are at about 70 to 80 mg TU per dose (e.g., 75 mg TU) or about 140 to 160 mg (e.g., 150 mg TU) per day TU. Typically, the doses of TU are administered with a meal.

It was surprisingly found that when employing TRT with a titration scheme as described herein (e.g., as described in the clinical trial in the Examples) that at the end of titration phase about 52% subjects ended up at 225 mg TU BID dose (same as starting dose), about 32% and about 16% ended up at 150 mg TU BID and 300 mg TU BID dose, respectively. Overall, about 38% subjects never required any titration and about 45% subjects underwent one titration. In subjects treated with a TRT and titration scheme as described herein, about 87% had serum testosterone C_{avg0-24h} within the normal range.

In some aspects and embodiments described herein, the TRT is indicated for testosterone replacement therapy in males for conditions associated with a deficiency or absence of endogenous testosterone. For example, (a) primary hypogonadism (congenital or acquired): testicular failure due to conditions such as cryptorchidism, bilateral torsion, orchitis, vanishing testis syndrome, orchidectomy, Klinefelter's syndrome, chemotherapy, or toxic damage from alcohol or heavy metals (these men usually have low serum testosterone concentrations and gonadotropins (FSH, LH) above the normal range) and (b) hypogonadotropic hypogonadism (congenital or acquired): gonadotropin or luteinizing hormone-releasing hormone (LHRH) deficiency or pituitary-hypothalamic injury from tumors, trauma, or radiation (these men usually have low testosterone serum concentrations but have gonadotropins in the normal or low range).

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

As used herein, the term “TU” refers to testosterone undecanoate.

The terms “serum testosterone” or “serum T levels,” “serum testosterone concentration,” “plasma testosterone concentration,” “testosterone concentration in the blood,” and “serum testosterone concentration,” are used interchangeably and refer to the “total” testosterone concentration which is the sum of the bioavailable testosterone including free and bound testosterone concentrations. Unless otherwise specified, these values are “observed” testosterone concentrations without adjusting or correcting for the base-line serum testosterone levels in the subject(s). As with any bio-analytical measure, for increased consistency, the method employed to measure initial serum testosterone levels should be consistent with the method used to monitor and re-measure serum testosterone levels during clinical testing and testosterone therapy for a subject. Unless otherwise stated, “testosterone concentration” refers to serum total testosterone concentration.

Average serum testosterone concentrations can be determined using methods and practices known in the art. For example, the average baseline plasma testosterone concentration of a human male is the arithmetic mean of the total plasma testosterone concentration determined on at least two consecutive time points that are reasonably spaced from each other, for example from about 1 hour to about 168 hours apart. In a particular case, the plasma testosterone concentration can be determined on at least two consecutive times that are about 12 hours to about 48 hours apart. In another particular method, the plasma testosterone concentration of the human male can be determined at a time between about 5 o'clock and about 11 o'clock in the morning. Further, the plasma testosterone concentration can be the determined by standard analytical procedures and methods available in the art, such as for example, automated or manual immunoassay methods, liquid chromatography or liquid chromatography-tandem mass spectrometry (LC-MSMS) etc.

As used herein, the term “AUCₜ₁₋ₜ₂” is the area under the curve of a plasma-versus-time graph determined for the analyte from the time “t1 to time t2”. Wherein t1 and t2 are times (in hours) post dosing. For Example, t1 could be 1 hour and t2 could be 2 hours.

As used herein, the term “C_{avg},” “Cₐᵥₑ,” or “C-average” are used interchangeably, and is determined as the AUCₜ₁₋ₜ₂ mean AUC divided by the time period (|t1−t2|). For example, C_{avg t0-t8} is the average plasma concentration over a period of 8 hours from t1=0 to t2=8 hours) post-dosing determined by dividing the AUCₜ₀₋ₜ₈ value by 8. Similarly, C_{avg t0-t12} is the average plasma concentration over a period of 12 hours post-dosing determined by dividing the AUCₜ₀₋ₜ₁₂ value by 12 (t1=0−t2=12). Similarly, C_{avg t12-t24} is the average plasma concentration over a period of 12 hours post-dosing determined by dividing the AUCₜ₁₂₋ₜ₂₄ value by 12 (t1=12−t2=24); C_{avg-t24} is the average plasma concentration over a period of 24 hours post-dosing determined by dividing the AUCₜ₀₋ₜ₂₄ value by 24 (t1=0-t2=24), and so on. Unless otherwise stated, all C_{avg} values are considered to be C_{avg-t24} and unless otherwise stated, all the time values are expressed in hours (h). For example, the term C_{avg t0-t24} denotes C_{avg} from time zero (0) to 24 hours post dosing. If no time is indicated then C_{avg} refer to C_{avg0-t24}.

As used herein, “Cₜ” refers to the serum concentration of testosterone at time “t” prior to or after administration of the dosage of the current invention. The time “t” is generally in hours, unless otherwise specified. For example, a Cₜ of “C_{(-2 to 0)} refers to serum testosterone concentration measured in sample collected between the time of about 2 hours before and just immediately prior to dosage administration to the subject tested. Similarly, Cₜ of “C_{(2 to 4)}” refers to serum testosterone concentration measured in sample collected between the time of about 2 hours and 4 hours after administration of a dosage to the subject tested.

As used herein, the term “Triton X100” or Triton “X-100” is a non-ionic detergent and refers to a composition as known as polyethylene glycol p-(1,1,3,3-tetramethylbutyl)-phenyl ether, octyl phenol ethoxylate, polyoxyethylene octyl phenyl ether, 4-octylphenol polyethoxylate, Mono 30, TX-100, t-octylphenoxypolyethoxyethanol, or Octoxynol-9 and associated with CAS NO. 9002-93-1.

Compositions or unit dosage forms of drug product can be considered to be “pharmaceutically equivalent”, if they meet three criteria: they contain the same active ingredient(s); they are of the same dosage form and route of administration; they are identical in strength or concentration. Typically pharmaceutical equivalent drug products may differ in characteristics such as shape, release mechanism, labeling (to some extent), scoring and excipients (including colors, flavors, and preservatives) although this list is not-limiting.

Compositions or unit dosages forms can be considered to be bioequivalent when administered under similar conditions to appropriate populations and using appropriate statistical analysis yield AUC₀₋₂₄ values (and/or AUC_{0-inf}) and/or Cmax values that are within 80% to 125% of the reference composition or unit dosage form (90% CI).

As used herein, the term “titration correlation window” refers to a period of time after administration of a single dose of testosterone undecanoate to a patient at steady state where the patient's serum testosterone levels correlate to C_{avg} or whether or not a dose adjustment should be made. In a specific definition, the titration correlation window is determined via clinical trial as is described in the Example.

As used herein, the term “representative population” refers to a number of subjects or patients from which a clinically meaningful decision can be made or determined. A clinically meaningful decision refers to a decision based on statistics.

In this specification, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term in the specification, like “comprising” or “including,” it is understood that direct support should be afforded also to “consisting essentially of” language as well as “consisting of” language as if stated explicitly and vice versa.

“The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, “enhanced,” “improved,” “performance-enhanced,” “upgraded,” and the like, when used in connection with the description of a composition, dosage form, regimen, or process, refers to a characteristic of the composition, dosage form, regimen, or process that provides measurably better form or function as compared to previously known compositions, dosage forms, regimens, or processes. This applies both to the form and function of individual components or ingredients in a composition, dosage form, regimen or process, as well as to such compositions, dosage forms, regiemens, or processes as a whole.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. However, it is to be understood that even when the term “about” is used in the present specification in connection with a specific numerical value, that support for the exact numerical value recited apart from the “about” terminology is also provided.

Reference will now be made in detail to specific embodiments of the invention. While the invention will be described in conjunction with such embodiments, it will be understood that it is not intended to limit the invention to those embodiments. To the contrary, it is intended to cover alternatives, variants, modifications, and equivalents as may be included within the spirit and scope of the invention as defined by the appended claims.

In one embodiment a unit dosage form is provided comprising: about 75 mg of testosterone undecanoate, about 112.5 mg of testosterone undecanoate, about 150 mg testosterone undecanoate, about 225 mg testosterone undecanoate or about 300 mg testosterone undecanoate; and a pharmaceutically acceptable carrier, wherein said pharmaceutical composition is for oral administration, or a pharmaceutically equivalent version thereof. In one aspect, when 1, 2, 3, or 4 unit dosage forms are administered with a meal twice a day to a hypogonadal male therapeutically effective C_{avg} serum testosterone levels are provided. In one aspect, the unit dosage form is a tablet or capsule. In one aspect, the unit dosage form has a pharmaceutically acceptable carrier which is a lipophilic additive, a hydrophilic additive, a solidifying agent or a combination thereof. In one aspect, the unit dosage form when tested using a USP type 2 apparatus in about 1000 mL 8% Triton X100 solution in water at 37.0±0.5 at 100 rpm releases at least 60% at 15 minutes and less than 100% at 15 minutes.

In one embodiment, a testosterone replacement therapy for twice daily dosing is provided said therapy comprising: (a) 2 different dose strength oral dosage forms having different amounts of testosterone undecanoate; (b) 3 dosing regimens providing for 3 different daily doses of testosterone undecanoate; (c) both (a) and (b); or (d) a pharmaceutically equivalent version thereof. In one aspect, the testosterone replacement therapy provides steady state serum levels of testosterone (C_{avg}) to a male having testosterone deficiency or in need of said therapy in the range of about 300 ng/dL to about 1140 ng/dL. In one aspect, the testosterone replacement therapy provides steady state serum levels of testosterone (C_{avg}) to a male having testosterone deficiency or in need of said therapy in the range of about 435 ng/dL to about 1140 ng/dL. In one aspect, the testosterone replacement therapy provides single dose Cₘₐₓ levels of serum testosterone at steady state to a population of males having testosterone deficiency, or in need of said therapy, of less than 2500 ng/dL in at least 95% of the population of males, less than 1500 ng/dL in at least 85% of the population of males; or a serum testosterone Cₘₐₓ of about 1800 ng/dL to about 2500 ng/dL in 10% or less of the population of males having testosterone deficiency. In one aspect, one of the dosage forms having different amounts of testosterone undecanoate has from about 140 to 160 mg testosterone undecanoate and the other dosage form has from about 215 mg to about 235 mg testosterone undecanoate. In one aspect, one of the dosage forms having different amounts of testosterone undecanoate has from about 145 to 155 mg testosterone undecanoate and the other dosage form has from about 220 mg to about 230 mg testosterone undecanoate. In one aspect, one of the dosage forms having different amounts of testosterone undecanoate has about 150 mg testosterone undecanoate and the other dosage form has about 225 mg testosterone undecanoate. In one aspect, one of the dosage forms having different amounts of testosterone undecanoate has from about 60 to 90 mg testosterone undecanoate and the other dosage form has from about 100 mg to about 130 mg testosterone undecanoate. In one aspect, one of the dosage forms having different amounts of testosterone undecanoate has from about 70 to 80 mg testosterone undecanoate and the other dosage form has from about 107 mg to about 118 mg testosterone undecanoate. In one aspect, one of the dosage forms having different amounts of testosterone undecanoate has about 75 mg testosterone undecanoate and the other dosage form has about 112.5 mg testosterone undecanoate. In one aspect, the testosterone replacement therapy has 3 dosing regimens providing for 3 different daily doses of testosterone undecanoate and provides for a first daily dose of about 275 mg to about 325 mg of testosterone undecanoate, a second daily dose of from about 425 mg to about 475 mg of testosterone undecanoate and a third daily dose of about 575 mg to about 625 mg of testosterone undecanoate. In one aspect, the testosterone replacement therapy provides for 3 different daily doses of testosterone undecanoate, providing for a first daily dose of about 300 mg of testosterone undecanoate a second daily dose of about 450 testosterone undecanoate and a third daily dose of about 600 mg of testosterone undecanoate. In one aspect, the testosterone replacement therapy comprises: 2 dosage forms, one having about 75 mg of testosterone undecanoate and the other dosage form having about 112.5 testosterone undecanoate; 3 dosing regimens, a first dosing regimen comprising administration of two dosage forms twice a day each dosage form having about 75 mg testosterone undecanoate, a second dosing regimen comprising administration of two dosage forms twice a day each dosage form having about 112.5 mg testosterone undecanoate, and a third dosing regimen comprising administration of four dosage forms twice a day each dosage form having about 75 mg testosterone undecanoate or a pharmaceutically equivalent version thereof. In one aspect, the testosterone replacement therapy comprises: 2 dosage forms, one having about 150 mg of testosterone undecanoate and the other dosage form having about 225 testosterone undecanoate; 3 dosing regimens, a first dosing regimen comprising administration of one dosage form twice a day each dosage form having about 225 mg testosterone undecanoate, a second dosing regimen comprising administration of one dosage form twice a day, the dosage form having about 150 mg testosterone undecanoate, and a third dosing regimen comprising administration of two dosage forms twice a day each dosage form having about 150 mg testosterone undecanoate (or one dosage form twice day each dosage form having about 300 mg testosterone undecanoate) or a pharmaceutically equivalent version thereof. As described herein, administration refers to administration to a subject in need of testosterone replacement e.g., a hypogonadal male or a male having low testosterone levels or a symptom thereof. In a specific aspect of this embodiment, the TRT accounts for or provides for dose titrations or adjustments. For example, if the subject's serum testosterone level about 3-6 hours after single dose administration is less than about 125 ng/dL, the dose of TU is adjusted up (e.g., an additional about 75 mg TU per dose or about 150 mg TU per day). In another example, if the subject's serum testosterone level about 3-6 hours after single dose administration is greater than about 1200 ng/dL, the dose of TU is adjusted down (e.g., decreased by about 75 mg TU per dose or about 150 mg TU per day). In yet another example, if the subject's serum testosterone is between about 125 ng/dL and 1200 ng/dL, the dose of TU remains the same.

In one embodiment, a testosterone replacement therapy for twice daily dosing is provided said therapy comprising: oral administration of a pharmaceutical composition comprising testosterone undecanoate at 3 dose titration levels or a pharmaceutically equivalent version thereof. As described herein, administration refers to administration to a subject in need of testosterone replacement e.g., a hypogonadal male or a male having low testosterone levels or a symptom thereof.

In one embodiment a therapy for treating a male having a baseline testosterone level of 300 ng/dL or less is provided said therapy comprising: (a) 2 unit dosage forms differing in amount of testosterone undecanoate; (b) 3 dosing regimens differing in total daily dose of testosterone undecanoate; (c) both (a) and (b); or (d) a pharmaceutically equivalent version thereof.

The testosterone replacement therapy as described in the above paragraphs can comprise an initial dosing regimen and a maintenance dosing regimen. The testosterone replacement therapy described as in the above paragraphs can comprise a dose titration determination. The testosterone replacement therapy as in the above paragraphs can comprise an initial dosing regimen which provides a daily dose of about 450 mg of testosterone undecanoate. The testosterone replacement therapy as described in the above paragraphs can comprise an initial dosing regimen which provides a daily dose of about 450 mg of testosterone undecanoate and a maintenance regimen daily dose of about 450 mg of testosterone undecanoate. The testosterone replacement therapy as in the above paragraphs can comprise an initial dosing regimen which provides a daily dose of about 450 mg of testosterone undecanoate and a maintenance regimen daily dose of about 600 mg of testosterone undecanoate. The testosterone replacement therapy as in the above paragraphs can comprise an initial dosing regimen which provides a daily dose of about 450 mg of testosterone undecanoate and a maintenance regimen daily dose of about 300 mg of testosterone undecanoate. The testosterone replacement therapy as described in the above paragraphs can comprise a dose titration determination which is made by measuring serum testosterone levels within a specified window of time after single dose administration of testosterone undecanoate said specified window of time correlating to C_{avg} values of 1140 ng/dL or greater; 300 ng/dL or lower; or between 300 ng/dL and 1140 ng/dL. The testosterone replacement therapy as in the above paragraphs can comprise a dose titration which is made by measuring serum testosterone levels within a specified window of time after single dose administration of testosterone undecanoate said specified window of time correlating to serum testosterone C_{avg} values of 1140 ng/dL or greater; 300 ng/dL or lower; or between 300 ng/dL and 1140 ng/dL and said serum testosterone levels within the specified window of time correlate to a serum testosterone C_{avg} of: 1140 ng/dL or more, a dose reduction is made; 300 ng/dL or lower, a dose increase is made; or between 300 ng/dL and 1140 ng/dL no dose adjustment is made. The therapy as in the above paragraphs can comprise a dose titration assessment for a patient receiving the therapy. For example, if the subject's serum testosterone level 3-6 hours after single dose administration with a meal at steady state is (a) less than about 125 ng/dL, the dose of TU is adjusted up (e.g., an additional 75 mg TU per dose or 150 mg TU per day); (b) greater than about 1200 ng/dL, the dose of TU is adjusted down (e.g., decreased by 75 mg TU per dose or 150 mg TU per day); or between about 125 ng/dL and 1200 ng/dL, the dose of TU remains the same.

In some aspects of the above embodiments, the dose titration assessment comprises determining a value of serum testosterone at from about two to eight hours after receiving a dose of testosterone undecanoate.

In some aspects of the above embodiments, a patient having (a) a low serum testosterone level at two to eight hours after receiving a single dose of testosterone undecanoate at steady state receives a higher dose of testosterone undecanoate; (b) a high serum testosterone level at two to eight hours after receiving a single dose of testosterone undecanoate at steady state receives a lower dose of testosterone undecanoate; (c) an intermediate serum testosterone level at two to eight hours after receiving a single dose of testosterone undecanoate at steady state receives an intermediate dose of testosterone undecanoate; or (d) a combination thereof. In a more specific aspect, if the subject's serum testosterone level about 3-6 hours after single dose administration is less than about 125 ng/dL, the dose of TU is adjusted up (e.g., an additional about 75 mg TU per dose or about 150 mg TU per day); if the subject's serum testosterone level about 3-6 hours after single dose administration is greater than about 1200 ng/dL, the dose of TU is adjusted down (e.g., decreased by about 75 mg TU per dose or about 150 mg TU per day); or if the subject's serum testosterone is between about 125 ng/dL and 1200 ng/dL, the dose of TU remains the same.

In some aspect of the above embodiments, the dose titration assessment comprises determining the serum testosterone level during a window period within two to eight hours after receiving a dose of testosterone undecanoate. In a more specific aspect, the dose titration assessment comprises determining the serum testosterone level during a window period within three to six hours after receiving a dose of testosterone undecanoate. In a more specific aspect, the dose titration assessment comprises determining the serum testosterone level during a window period within three to six hours after receiving a dose of testosterone undecanoate.

In some aspect of the above embodiments, the window period is the titration correlation window for a representative population.

In one embodiment, a testosterone replacement therapy for twice daily dosing is provided said therapy comprising: 3 dose titration levels for daily doses of testosterone undecanoate providing a lower dose, an intermediate dose and a higher dose or a pharmaceutically equivalent version thereof. In one aspect, the higher and lower dose is within about 40% of the intermediate dose. In one aspect, a patient receiving the therapy has a dose titration assessment. In one aspect, the dose titration assessment comprises determining a value of serum testosterone at from about two to eight hours after receiving a dose of testosterone undecanoate. In one aspect, a patient having (a) a low serum testosterone level at two to eight hours after receiving a single dose of testosterone undecanoate at steady state receives a higher dose of testosterone undecanoate; (b) a high serum testosterone level at two to eight hours after receiving a single dose of testosterone undecanoate at steady state receives a lower dose of testosterone undecanoate; (c) an intermediate serum testosterone level after receiving a single dose of testosterone undecanoate at steady state receives an intermediate dose of testosterone undecanoate; or (d) a combination thereof. In one aspect, the dose titration assessment comprises determining the serum testosterone level during a window period within two to eight hours after receiving a dose of testosterone undecanoate. In one aspect, the window period is the titration is the correlation window for a representative population. In one aspect, the testosterone replacement therapy provides singledose Cₘₐₓ levels of serum testosterone at steady state level to a population of males having testosterone deficiency, or in need of said therapy, of (a) less than 2500 ng/dL in at least 95% of the population of males; (b) less than 1500 ng/dL in at least 85% of the population of males; a serum testosterone Cₘₐₓ of about 1800 ng/dL to about 2500 ng/dL in 10% or less of the subjects in the group; or a combination thereof. In a more specific aspect, if the subject's serum testosterone level about 3-6 hours after single dose administration is less than about 125 ng/dL, the dose of TU is adjusted up (e.g., an additional about 75 mg TU per dose or about 150 mg TU per day); if the subject's serum testosterone level about 3-6 hours after single dose administration is greater than about 1200 ng/dL, the dose of TU is adjusted down (e.g., decreased by about 75 mg TU per dose or about 150 mg TU per day); or if the subject's serum testosterone is between about 125 ng/dL and 1200 ng/dL, the dose of TU remains the same.

In one embodiment, a therapy for treating a male having a baseline serum testosterone level of 300 ng/dL or less is provided said therapy comprising: (a) 2 oral dosage forms having different amounts of testosterone undecanoate; (b) 3 dosing regimens providing different daily doses of testosterone undecanoate; (c) both (a) and (b); or (d) a pharmaceutically equivalent version thereof. In one aspect, the higher and lower daily doses are within about 40% of the intermediate dose. In one aspect, the therapy provides singledose Cₘₐₓ levels of serum testosterone at steady state levels to a population of males having testosterone deficiency, or in need of said therapy, of (a) less than 2500 ng/dL in at least 95% of the population of males; (b) less than 1500 ng/dL in at least 85% of the population of males; a serum testosterone Cₘₐₓ of about 1800 ng/dL to about 2500 ng/dL in 10% or less of the subjects in the group; or a combination thereof. In one aspect, a patient receiving the therapy has a dose titration assessment. In one aspect, the dose titration assessment comprises determining a value of serum testosterone at from about two to eight hours after receiving a dose of testosterone undecanoate. In one aspect, a patient having (a) a low serum testosterone level at two to eight hours after receiving a single dose of testosterone undecanoate at steady state receives a higher dose of testosterone undecanoate; (b) a high serum testosterone level at two to eight hours after receiving a single dose of testosterone undecanoate at steady state receives a lower dose of testosterone undecanoate; (c) an intermediate serum testosterone level after receiving a single dose of testosterone undecanoate at steady state an intermediate dose of testosterone undecanoate; or (d) a combination thereof. In one aspect, the dose titration assessment comprises determining the serum testosterone level during a window period within two to eight hours after receiving a dose of testosterone undecanoate. In one aspect, the window period is the titration correlation window for a representative population. In a more specific aspect, if the subject's serum testosterone level about 3-6 hours after single dose administration is less than about 125 ng/dL, the dose of TU is adjusted up (e.g., an additional about 75 mg TU per dose or about 150 mg TU per day); if the subject's serum testosterone level about 3-6 hours after single dose administration is greater than about 1200 ng/dL, the dose of TU is adjusted down (e.g., decreased by about 75 mg TU per dose or about 150 mg TU per day); or if the subject's serum testosterone is between about 125 ng/dL and 1200 ng/dL, the dose of TU remains the same.

In one embodiment, a unit dosage form is provided comprising: about 75 mg of testosterone undecanoate or about 112.5 mg of testosterone undecanoate; and a pharmaceutically acceptable carrier, wherein said pharmaceutical composition is for oral administration, or a pharmaceutically equivalent version thereof. The unit dosage forms of this embodiment may be used in any of the therapies described herein. In one aspect, when 2, 3, or 4 unit dosage forms are administered with a meal twice a day to a hypogonadal male therapeutically effective serum testosterone C_{avg} levels are provided. In one aspect, the unit dosage form provides a serum testosterone C_{avg} of 400-800 ng/dL when administered as 2, 3, or 4 unit dosage forms twice daily to a hypogonadal male.

In some embodiments, the testosterone replacement therapy with testosterone undecanoate is administered with a meal.

Thus, provided herein in one embodiment, is a testosterone replacement therapy for twice daily dosing said therapy comprising: 3 dose titration levels for daily doses of testosterone undecanoate providing a lower dose, an intermediate dose and a higher dose; or a pharmaceutically equivalent version thereof. In one aspect, the testosterone replacement therapy has a higher and lower dose which within about 40% of the intermediate dose. In one aspect, the patient receiving the therapy has a dose titration assessment. In one aspect, the dose titration assessment comprises determining a value of serum testosterone at from about two to eight hours after receiving a single dose of testosterone undecanoate at steady state. In one aspect, a patient having (a) a low serum testosterone level at two to eight hours after receiving a single dose of testosterone undecanoate receives a higher dose of testosterone undecanoate; (b) a high serum testosterone level at two to eight hours after receiving a single dose of testosterone undecanoate receives a lower dose of testosterone undecanoate; (c) an intermediate serum testosterone level at two to eight hours after receiving a single dose of testosterone undecanoate receives an intermediate dose of testosterone undecanoate; or (d) a combination thereof. In one aspect, the dose titration assessment comprises determining the serum testosterone level during a window period within two to eight hours after receiving a single dose of testosterone undecanoate at steady state. In one aspect, the window period for the titration is the correlation window for a representative population. In one aspect, the testosterone replacement therapy provides a single dose Cₘₐₓ levels of serum testosterone at steady state level to a population of males having testosterone deficiency, or in need of said therapy, of (a) less than 2500 ng/dL in at least 95% of the population of males; (b) less than 1500 ng/dL in at least 85% of the population of males; a serum testosterone Cₘₐₓ of about 1800 ng/dL to about 2500 ng/dL in 10% or less of the subjects in the group; or a combination thereof.

Provided herein in one embodiment is a therapy for treating a male having a baseline serum testosterone level of 300 ng/dL or less said therapy comprising: (a) 2 oral dosage forms having different amounts of testosterone undecanoate; (b) 3 dosing regimens; (c) both (a) and (b); or (d) a pharmaceutically equivalent version thereof. In one aspect, the three dosing regimens involve three different daily doses of TU with the higher and lower doses of TU being within about 40% of the intermediate dose. In one aspect of this embodiment, the therapy provides singledose Cₘₐₓ levels of serum testosterone at steady state levels to a population of males having testosterone deficiency or in need of said therapy, of (a) less than 2500 ng/dL in at least 95% of the population of males; (b) less than 1500 ng/dL in at least 85% of the population of males; a serum testosterone Cₘₐₓ of about 1800 ng/dL to about 2500 ng/dL in 10% or less of the subjects in the group; or a combination thereof. In one aspect, a patient receiving the therapy has a dose titration assessment. In one aspect, the dose titration assessment comprises determining a value of serum testosterone at from about two to eight hours after receiving a single dose of testosterone undecanoate at steady state. In one aspect, a patient having (a) a low serum testosterone level at two to eight hours after receiving a single dose of testosterone undecanoate receives a higher dose of testosterone undecanoate; (b) a high serum testosterone level at two to eight hours after receiving a single dose of testosterone undecanoate receives a lower dose of testosterone undecanoate; (c) an intermediate serum testosterone level at two to eight hours after receiving a single dose of testosterone undecanoate receives an intermediate dose of testosterone undecanoate; or (d) a combination thereof. In one aspect, the dose titration assessment comprises determining the serum testosterone level during a window period within two to eight hours after receiving a dose of testosterone undecanoate. In one aspect, the window period is the titration correlation window for a representative population.

Provided herein in some embodiments is a unit dosage form comprising: about 75 mg of testosterone undecanoate or about 112.5 mg of testosterone undecanoate; and a pharmaceutically acceptable carrier, wherein said pharmaceutical composition is for oral administration, or a pharmaceutically equivalent version thereof. In one aspect, when 2, 3, or 4 unit dosage forms are administered with a meal twice a day to a hypogonadal male therapeutically effective serum testosterone C_{avg} levels are provided. In one aspect, the unit dosage form provides serum testosterone C_{avg} of 400-800 ng/dL when administered as 2, 3, or 4 unit dosage forms twice daily to a hypogonadal male.

Typically, the unit dosage forms contain an amount of TU as described herein and one or more pharmaceutically acceptable carriers e.g., additives. The unit dosage forms usually contain a lipophilic additive. The dosage forms can also contain a hydrophilic additive, a solidifying agent, or one or more other additives.

In another embodiment, the lipophilic additive can include a lipophilic surfactant.

As used herein a surfactant is considered to be a lipophilic surfactant when it has an HLB value of 10 or less. Various lipophilic surfactants can be used including, but not limited to mono-, di-glycerides of fatty acids like glyceryl monolinoleate (e.g., Maisine® 35-1), mono- and di glycerides of caprylic, capric acid (e.g., Capmul® MCM), glyceryl monooleate, reaction mixtures of alcohols or polyalcohols with a variety of natural and/or hydrogenated oils such as PEG-5 hydrogenated castor oil, PEG-7 hydrogenated castor oil, PEG-9 hydrogenated castor oil, PEG-6 corn oil (e.g., Labrafil® M 2125 CS), PEG-6 almond oil (e.g., Labrafil® M 1966 CS), PEG-6 apricot kernel oil (e.g., Labrafil® M 1944 CS), PEG-6 olive oil (e.g., Labrafil® M 1980 CS), PEG-6 peanut oil (e.g., Labrafil® M 1969 CS), PEG-6 hydrogenated palm kernel oil (e.g., Labrafil®. M 2130 BS), PEG-6 palm kernel oil (e.g., Labrafil® M 2130 CS), PEG-6 triolein (e.g., Labrafil® M 2735 CS), PEG-8 corn oil (e.g., Labrafil® WL 2609 BS), PEG-20 corn glycerides (e.g., Crovol® M40), PEG-20 almond glycerides (e.g., Crovol® A40), lipophilic polyoxyethylene-polyoxypropylene block co-polymers (e.g., Pluronic® L92, L101, L121 etc.); propylene glycol fatty acid esters, such as propylene glycol monolaurate (e.g., Lauroglycol FCC), propylene glycol ricinoleate (e.g. Propymuls), propylene glycol monooleate (e.g., Myverol P-O6), propylene glycol dicaprylate/dicaprate (e.g., Captex® 200), and propylene glycol dioctanoate (e.g., Captex® 800), propylene glycol mono-caprylate (e.g., Capryol® 90); propylene glycol oleate (e.g., Lutrol OP2000); propylene glycol myristate; propylene glycol mono stearate; propylene glycol hydroxy stearate; propylene glycol ricinoleate; propylene glycol isostearate; propylene glycol mono-oleate; propylene glycol dicaprylate/dicaprate; propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (e.g., Arlacel® 186); sterol and sterol derivatives such as cholesterol, sitosterol, phytosterol, phytosterol fatty acid esters, PEG-5 soya sterol, PEG-10 soya sterol, PEG-20 soya sterol, and the like; glyceryl palmitostearate, glyceryl stearate, glyceryl distearate, glyceryl monostearate, or a combination thereof; sorbitan fatty acid esters such as sorbitan monolaurate (e.g., Arlacel 20), sorbitan monopalmitate (e.g., Span-40), sorbitan monooleate (e.g., Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; fatty acids such as capric acid, caprylic acid, oleic acid, linoleic acid, myristic acid, menthol, menthol derivatives, lecithin, phosphatidyl choline, bile salts, and the like, and mixtures thereof. It is important to note that some lipophilic surfactants may also function as the solubilizer component of the compositions and oral dosage forms.

In one embodiment, the lipophilic surfactant can be selected from the group consisting of glyceryl monolinoleate (e.g., Maisine® 35-1), mono- and di glycerides of caprylic, capric acid (e.g., Capmul® MCM), glyceryl monooleate, propylene glycol mono caprylate, propylene glycol oleate, propylene glycol monostearate, propylene glycol monolaurate, propylene glycol mono-oleate, propylene glycol dicaprylate/dicaprate, sorbitan monooleate, PEG-5 hydrogenated castor oil, PEG-7 hydrogenated castor oil, PEG-9 hydrogenated castor oil, PEG-6 corn oil, PEG-6 almond oil, PEG-6 apricot kernel oil, PEG-6 olive oil, PEG-6 peanut oil, PEG-6 hydrogenated palm kernel oil, sorbitan monolaurate (e.g., Arlacel 20), sorbitan monopalmitate, sorbitan monooleate, sorbitan monostearate, sorbitan tristearate, sorbitan monolaurate, sorbitan monopalmitate, sorbitan monooleate, sorbitan trioleate, sorbitan sesquioleate, sorbitan tristearate, sorbitan monoisostearate, and combinations thereof. In some embodiments, the lipophilic surfactants can comprise at least about 10, 20, 30, 40, 50, 60, 70, 80, or 90 wt % of the total pharmaceutically acceptable carrier. It should be noted that the combinations of two or more lipophilic surfactants from the same or different classes therein are also within the scope of this invention and are together can be referred to as the lipophilic surfactant, unless otherwise stated. In one embodiment, the hydrophilic additive can be a hydrophilic surfactant. A surfactant is considered to be a hydrophilic surfactant when it has an HLB value of greater than 10. Non-limiting examples of hydrophilic surfactants include non-ionic surfactants, ionic surfactants and zwitterionic surfactants. Specifically the hydrophilic surfactants suitable for the current invention include, but not limited to alcohol-oil transesterification products; polyoxyethylene hydrogenated vegetable oils; polyoxyethylene vegetable oils; alkyl sulphate salts, dioctyl sulfosuccinate salts; polyethylene glycol fatty acids esters; polyethylene glycol fatty acids mono- and di-ester mixtures; polysorbates, polyethylene glycol derivatives of tocopherol and the like. It should be noted that the combinations of two or more hydrophilic surfactants from the same or different classes are within the scope of this invention and are together can be referred to as the hydrophilic surfactant unless explicitly specified. In one embodiment, the hydrophilic additive can be a hydrophilic surfactant. Non-limiting examples of hydrophilic surfactants can include PEG-8 caprylic/capric glycerides, lauroyl macrogol-32 glyceride, stearoyl macrogol glyceride, PEG-40 hydrogenated castor oil, PEG-35 castor oil, sodium lauryl sulfate, sodium dioctyl sulfosuccinate, polyethylene glycol fatty acids mono- and di-ester mixtures, polysorbate 80, polysorbate 20, polyethylene glycol 1000 tocopherol succinate, phytosterols, phytosterol fatty acid esters, and mixtures thereof.

Suitable additives utilized in various embodiments described herein include, by way of non-limiting example, adsorbing agents, anti-adherents, anticoagulants, antifoaming agents, antioxidants, anti-caking agents, anti-static agents, binders, bile acids, bufferants, bulking agents, chelating agents, coagulants, colorants, co-solvent, opaquants, congealing agents, coolants, cryoprotectants, diluents, dehumidifying agents, desiccants, desensitizers, disintegrants, dispersing agents, enzyme inhibitors, glidants, fillers, hydrating agent, super disintegrants, gums, mucilages, hydrogen bonding agents, enzymes, flavorants, humectants, humidifying agents, lubricant oils, ion-exchange resins, lubricants, plasticizers, pH modifying agents, preservatives, solidifying agent, solvents, solubilizers, spreading agent sweeteners, stabilizers, surface area enhancing agents, suspending agent, thickeners, viscosity increasing agents, waxes and mixtures thereof.

Some non-limiting examples of the additives suitable for the present disclosure may be: alcohols and/or polyols (e.g., ethanol, isopropanol, butanol, benzyl alcohol, ethylene glycol, propylene glycol, glycerol, sorbitol, mannitol, dimethyl isosorbide, polyethylene glycol, fatty acid alcohol, vinyl alcohol polypropylene glycol, polyvinylalcohol, tocopherols, cellulose cyclodextrins, other derivatives, forms, mixtures thereof, or the like); ethers of polyethylene glycols having an average molecular weight of about 200 to about 20,000 (e.g., tetrahydrofurfuryl alcohol PEG ether, methoxy PEG, or the like); amides (e.g., 2-pyrrolidone, 2-piperidone, 8-caprolactam, N-alkylpyrrolidone, N-hydroxyalkylpyrrolidone, N-alkylpiperidone, N-alkylcaprolactam, dimethylacetamide, polyvinylpyrrolidone and the like); esters (e.g., ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, 8-caprolactone and isomers thereof, 6-valerolactone and isomers thereof, y-butyrolactone and isomers thereof; and other additives known in the art, such as dimethyl acetamide, dimethyl isosorbide, N-methylpyrrolidones, monooctanoin, diethylene glycol monoethyl ether, or the like); amino acids (e.g., p-aminobenzamidine, sodium glycocholate) mesylate; amino acids and modified amino acids (e.g., aminoboronic acid derivatives and n-acetylcysteine; peptides and modified peptides (e.g., bacitracin, phosphinic acid dipeptide derivatives, pepstatin, antipain, leupeptin, chymostatin, elastin, bestatin, phoshporamindon, puromycin, cytochalasin potatocarboxy peptidase inhibitor, amastatin, or the like); polypeptide protease inhibitors; mucoadhesive polymers (e.g., polyacrylate derivatives, chitosan, cellulosics, chitosan-EDTA, chitosan-EDTA-antipain, polyacrylic acid, carboxymethyl cellulose, etc.,) or the like; or combinations thereof.

Some more examples of suitable additives for compositions and/or dosage forms described herein include, by way of non-limiting example, talc, magnesium stearate, silica (e.g., fumed silica, micronized silica, magnesium aluminum silicate, etc.) and/or derivatives, polyethylene glycols, surfactants, waxes, oils, cetyl alcohol, polyvinyl alcohol, stearic acid, stearic acid salts, stearic acid derivatives, starch, hydrogenated vegetable oils, hydrogenated castor oils, sodium benzoate, sodium acetate, leucine, PEG, alkyl sulfate salts; acetylated monoglycerides; long-chain alcohols; silicone derivatives; butylated hydroxy toluene (BHT), butylated hydroxyl anisole (BHA), gallic acid, propyl gallate, ascorbic acid, ascorbyl palmitate, 4-hydroxymethyl-2,6-di-tert-butyl phenol, dry starch, dry sugars, polyvinyl pyrrolidones, starch paste, methacrylic copolymers, bentonite, sucrose, polymeric cellulose derivatives, shellac, sugar syrup; corn syrup; polysaccharides, acacia, tragacanth, guar gum, xanthan gums; alginates; gelatin; gelatin hydrolysate; agar; sucrose; dextrose; PEG, vinyl pyrrolidone copolymers, poloxamers; pregelatinized starch, sorbitol, glucose); acetic acid, hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, vinegar, pharmaceutically acceptable bases, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamin; salt of a pharmaceutically acceptable cation and an anion; EDTA and EDTA salts; titanium dioxide, food dyes, lakes, natural vegetable colorants, iron oxides, silicates, sulfates, magnesium hydroxide and aluminum hydroxide; halogenated hydrocarbons, trichloro ethane, trichloro ethylene, dichloromethane, fluorotrichloromethane, diethylether, trehalose, phosphates, citric acid, tartaric acid, gelatin, dextran and mannitol, lactose, mannitol, sodium chloride, potassium chloride, spray-dried lactose, hydrolyzed starches, directly compressible starch, microcrystalline cellulose, cellulosic derivatives, sorbitol, sucrose, sucrose-based materials, calcium sulfate, dibasic calcium phosphate, dextrose, croscarmellose sodium, starch, starch derivatives, clays, gums, cellulose, cellulose derivatives, alginates, crosslinked polyvinylpyrrolidone, sodium starch glycolate and microcrystalline cellulose, magnesium oxide, magnesium carbonates; desensitizers, spray-dried flavors, essential oils, ethyl vanillin, styrene/divinyl benzene copolymers, quaternary ammonium compounds, polyethylene glycol, citrate esters (such as triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl sebacate, ascorbic acid, boric acid, sorbic acid, benzoic acid, and salts thereof, parabens, phenols, benzyl alcohol, and quaternary ammonium compounds; alcohols, ketones, esters, chlorinated hydrocarbons water; sweeteners (e.g., maltose, sucrose, glucose, sorbitol, glycerin and dextrins, aspartame, saccharine, saccharine salts, glycyrrhizin), viscosity modifiers, sugars, polyvinylpyrrolidone, cellulosics, polymers, gums and/or alginates.

In one embodiment, additives may also be materials such as proteins (e.g., collagen, gelatin, Zein, gluten, mussel protein, lipoprotein); carbohydrates (e.g., alginates, carrageenan, cellulose derivatives, pectin, starch, chitosan); gums (e.g., xanthan gum, gum Arabic); spermaceti; natural or synthetic waxes; carnauba wax; fatty acids (e.g., stearic acid, hydroxystearic acid); fatty alcohols; sugars; shellacs, such as those based on sugars (e.g., lactose, sucrose, dextrose) or starches; polysaccharide-based shellacs (e.g., maltodextrin and maltodextrin derivatives, dextrates, cyclodextrin and cyclodextrin derivatives); cellulosic-based polymers (e.g., ethyl cellulose, methyl cellulose, microcrystalline cellulose, sodium carboxymethyl cellulose, hydroxypropylmethyl cellulose, ethyl cellulose, hydroxypropyl cellulose, HPMC acid succinates, cellulose acetate, cellulose nitrate, cellulose acetate butyrate, cellulose acetate trimellitate, carboxymethylethyl cellulose, hydroxypropylmethyl cellulose phthalate), shellacs; inorganics, such as dicalcium phosphate, hydroxyapatite, tricalcium phosphate, talc and titania; polyols, such as mannitol, xylitol and sorbitol; polyethylene glycol esters; and polymers, such as alginates, poly(lactide coglycolide), gelatin, crosslinked gelatin, and agar-agar. Non-limiting examples of compounds (e.g., additives) that can be used as at least a part of the pharmaceutically acceptable carrier include without limitation celluloses; dextrins, gums, carbomers, methacrylates, sugars, lactoses, inorganic carbonates, oxides, chlorides, sulphates and the like; salts of calcium; salts of magnesium; salts of fatty acids; inorganic and organic acids, bases and salts; propylene glycol; glycerols; fatty acids; fatty alcohols; fatty acid esters; glycerol esters; mono-, di- or triglycerides; edible oils; omega oils; vegetable oils, hydrogenated vegetable oils; partially or fully hydrogenated vegetable oils; glycerol esters of fatty acids; waxes; alcohols; gelatin; polyethylene glycol; polyethylene oxide co-polymers; silicates; antioxidants, tocopherols, sugar stearates, starches, shellac, resins, proteins, acrylates; methyl copolymers; polyvinyl alcohol; starch; phthalates; and combinations thereof. In one embodiment, the additive may include at least one component selected from celluloses, dextrins, gums, carbomers, methacrylates, inorganic carbonates, salts of calcium, salts of magnesium, fatty acids, fatty acid esters, gelatin, lactoses, polyethylene glycol, polyethylene oxide co-polymers, silicates, partially hydrogenated vegetable oils, fully hydrogenated vegetable oils, waxes, antioxidants, tocopherol, sugar stearates, starches, shellac, resins, proteins, and combinations thereof.

In another embodiment, the additive may include at least one component selected from celluloses, dextrins, gums, carbomers, methacrylates, sugars, lactoses, inorganic carbonates, salts of calcium, salts of magnesium, salts of fatty acids, inorganic and organic acids, bases and salts, propylene glycol, glycerols, fatty acids, fatty alcohols, fatty acid esters, glycerol esters, mono-glycerol esters of fatty acids, di-glycerol esters of fatty acids, mixtures of mono-glycerol and di-gylcerol esters of fatty acids, omega oils, waxes, alcohols, gelatin, polyethylene glycol, polyethylene oxide co-polymers, silicates, antioxidants, tocopherol, sugar stearates, starches, shellac, resins, proteins, acrylates, methyl copolymers, polyvinyl alcohol, starch, phthalates, and combinations thereof.

Non-limiting examples of additives as release modulators that may be used include lipophilic resins; ethyl cellulose (EC), methylethyl cellulose (MEC), carboxymethyl ethylcellulose (CMEC), hydroxyethyl cellulose (HEC), cellulose acetate (CA), cellulose propionate (CPr), cellulose butyrate (CB), cellulose acetate butyrate (CAB), cellulose acetate phthalate (CAP), cellulose acetate trimellitate (CAT), hydroxypropyl methyl cellulose phthalate (HPMCP), hydroxypropyl methyl cellulose acetate succinate (HPMCAS), hydroxypropyl methyl cellulose acetate trimellitate (HPMCAT), ion-exchange resin; poloxamers; and ethylhydroxy ethylcellulose (EHEC) tocopherol; shellac; and combinations thereof. Non-limiting examples of lipidic lipophilic release modulators include fatty acids; mono-, di-, tri-esters of fatty acids with glycerol; sucrose esters with fatty acids; cetyl alcohol; stearic acid; glyceryl monostearate; glyceryl distearate; glyceryl tristearate; glyceryl palmitostearate; hydrogenated castor oil; butyl and glycol esters of fatty acids; oleic acid; cetyl alcohol; stearyl alcohol; cetostearyl alcohol; hydrogenated vegetable oil; waxes; bees wax; lard; omega fatty acid esters; hydrogenated soybean oil; hydrogenated vegetable oil; hydrogenated cottonseed and castor oil; partially hydrogenated soybean oil; partially hydrogenated castor oil; partially soy and cottonseed oil; phospholipids; hydrogenated oils, and their derivatives and combinations thereof.

In a more specific aspect of this embodiment, the testosterone undecanoate as described in this paragraph are further part of a composition having one or more components chosen from the following:

(A) octadecanoic acid, (9Z)-octadec-9-enoic acid, (9Z,12Z)-9,12-octadecadienoic acid, or hexadecanoic acid;
(B) a mono-, di-, or tri-propane-1,2,3-triol ester of (A);
(C) a combination of mono-, di-, or tri-propane-1,2,3-triol esters of (A);
(D) a combination of one or more of (A)-(C);
(E) (2S,5R)-2-Isopropyl-5-methylcyclohexanone, acetic acid [(1R,2S,5R)-2-isopropyl-5-methylcyclohexyl] ester; (1R,2S,5R)-2-isopropyl-5-methylcyclohexanol or a combination thereof;
(F) 2-isopropyl-5-methylcyclohexanol, 2-Isopropyl-5-methylcyclohexanone, acetic acid [(2-isopropyl-5-methylcyclohexyl] ester or a combination thereof.
(G) polyoxyethylated oil;
(H) polyoxyethylated hydrogenated vegetable oil;
(I) polyoxyethylated hydrogenated vegetable oil;
(J) polyoxyethylated hydrogenated castor oil;
(K) H—(O—CH₂—CH₂)ₙ—OH where n is an integer from 5 to 600;
(L) a branched; star, or comb analog (in this specific context analog refers to a molecule having the same molecular weight or average molecular weight) of H—(O—CH₂—CH₂)ₙ—OH where n is an integer from 5 to 600; and
(M) polvinylpyrrolidone, hydroxypropyl methylcellulose, hydroxypropyl cellulose, cellulose acetate phthalate, polyvinyl acetate phthalate, polyethylene oxide, poly(acrylic acid), polymethyacrylate, poly(ethylene oxide)-polypropylene oxide)-poly(ethylene oxide), polyvinyl alcohol, polystyrenesulfonic acid, polyvinylpyrrolidone-co-polyvinyl acetate, polyether polyol, carboxymethylcellulose, methylcellulose, hydroxyethyl cellulose, hydroxypropylmethyl cellulose phthalate, hydroxypropylmethyl cellulose acetate succinate, or a combination thereof.

Specific formulations can be as follows. It is noted that the amounts and percentages of components can be modified in view of the TRT described herein to yield a robust efficacious and safe TRT. In specific aspects, the amounts of TU in mg in the unit dosage forms are as described elsewhere in this application as ranges or specific amounts to provide the daily doses with 2 to 8 unit dosage (e.g., 2, 3, 4, 5, 6, 7, or 8) forms per day.

Shown below are various compositions suitable for oral administration as described herein. In these Examples the amount of excipient adds up to 100% (does not include the API) and the API weight percent is the final weight percent in the pharmaceutical composition.

TABLE A

| Component | Composition No. | | | | | | |
|---|---|---|---|---|---|---|---|
| (w/w %) | 1 | 2 | 3 | 4 | 5 | 6 | 7 |
| API | 22 | 23 | 24 | 26 | 28 | 30 | 32 |
| Excipient 1 | 35-80 | 35-80 | 35-80 | 35-80 | 35-80 | 35-80 | 35-80 |
| (e.g., liquid | | | | | | | |
| carrier) | | | | | | | |
| Excipient 2 | 1-40 | 1-40 | 1-40 | 1-40 | 1-40 | 1-40 | 1-40 |
| (e.g., additive) | | | | | | | |
| Excipient 3 | 0-20 | 0-20 | 0-20 | 0-20 | 0-20 | 0-20 | 0-20 |
| (e.g., hydrophilic | | | | | | | |
| additive) | | | | | | | |
| Excipient 4 | 0.01-3 | 0.01-3 | 0.01-3 | 0.01-3 | 0.01-3 | 0.01-3 | 0.01-3 |
| (e.g., anti- | | | | | | | |
| oxidant) | | | | | | | |
| Additional | qs | qs | qs | qs | qs | qs | qs |
| Excipients | | | | | | | |
| (e.g., other | | | | | | | |
| pharmaceutically | | | | | | | |
| acceptable | | | | | | | |
| excipients) | | | | | | | |

The API in this example in specific compositions is TU. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid; H—(O—CH₂—CH₂)ₙ—OH where n is an integer from 3 to 900; octadecanoic acid; (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene; or a combination thereof. Excipient 3 in specific compositions is a polyoxylated hydrogenated vegetable oil. Excipient 4 in specific compositions is ascorbyl palmitate. These compositions can be filled into soft gel or hard gel capsules depending on its flowability at the temperatures useful for making these dosage forms.

Shown below are various compositions suitable for oral administration as described herein. In these Examples the amount of excipient adds up to 100% (does not include the API) and the API weight percent is the final weight percent in the pharmaceutical composition

TABLE B

| Component | Composition No. | | | | | | | | |
|---|---|---|---|---|---|---|---|---|---|
| (w/w %) | 10 | 11 | 12 | 13 | 14 | 15 | 16 | 17 | 18 |
| API | 23 | 24 | 25 | 26 | 27 | 28 | 29 | 30 | 31 |
| Excipient 1 | 40-70 | 30-70 | 40-70 | 40-70 | 40-70 | 40-70 | 40-70 | 30-70 | 40-70 |
| (e.g., C14-C20 | | | | | | | | | |
| fatty acid) | | | | | | | | | |
| Excipient 2 | 0.5-20 | | | 1-20 | | | 1-20 | | |
| (e.g., glyceryl | | | | | | | | | |
| palmitostearate) | | | | | | | | | |
| Excipient 3 | | 0.5-30 | | | 5-35 | | | 10-30 | |
| Excipient 4 | | | 0.5-15 | | | 1-12 | | | 2-11 |
| (e.g., polyethylene | | | | | | | | | |
| glycol (high | | | | | | | | | |
| molecular weight)) | | | | | | | | | |
| Excipient 4 | 0.01-3 | 0.01-3 | 0.01-3 | 0.01-3 | 0.01-3 | 0.01-3 | 0.01-3 | 0.01-3 | 0.01-3 |
| (e.g., anti-oxidant | | | | | | | | | |
| (ascorbyl | | | | | | | | | |
| palmitate) | | | | | | | | | |
| Additional | qs | qs | qs | qs | qs | qs | qs | qs | qs |
| Excipients | | | | | | | | | |

The API in these examples in specific compositions is TU. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid, octadecanoic acid or a combination thereof. Excipient 3 in specific compositions is (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene. Excipient 4 in specific compositions is H—(O—CH₂—CH₂)ₙ—OH where n is an integer from 3 to 900 (e.g., PEG having an average molecular weight in the range of 2000-12000). These compositions can be filled into soft gel or hard gel capsules depending on its flowability at the temperatures useful for making these dosage forms. These compositions may include a hydrophilic additive.

Shown below are various compositions suitable for oral administration as described herein. In these Examples the amount of excipient adds up to 100% (does not include the API) and the API weight percent is the final weight percent in the pharmaceutical composition.

TABLE C

| Component | Composition No. | | | | | | | | |
|---|---|---|---|---|---|---|---|---|---|
| (w/w %) | 19 | 20 | 21 | 22 | 23 | 24 | 25 | 26 | 27 |
| API | 23 | 24 | 25 | 26 | 27 | 28 | 29 | 30 | 31 |
| Excipient 1 | 40-90 | 40-90 | 40-90 | 40-90 | 40-90 | 40-90 | 40-90 | 40-90 | 40-90 |
| Excipient 2 | 1-20 | | 1-20 | | 1-20 | | 1-20 | | 1-20 |
| Excipient 3 | | 1-10 | | 1-10 | | 1-10 | | 1-10 | |
| Excipient 4 | 0-25 | 0-25 | 0-25 | 0-25 | 0-25 | 0-25 | 0-25 | 0-25 | 0-25 |
| Additional | qs | qs | qs | qs | qs | qs | qs | qs | qs |
| Excipients | | | | | | | | | |

The API in this example in specific compositions is TU. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid, octadecanoic acid or a combination thereof. Excipient 3 in specific compositions is H—(O—CH₂—CH₂)ₙ—OH where n is an integer from 3 to 900 (e.g., PEG having an average molecular weight in the range of 2000-12000). Excipient 4 in specific compositions is (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene. These compositions can be filled into soft gel or hard gel capsules depending on its flowability at the temperatures useful for making these dosage forms.

Shown below are various compositions suitable for oral administration as described herein. In these Examples the amount of excipient adds up to 100% (does not include the API) and the API weight percent is the final weight percent in the pharmaceutical composition.

TABLE D

| Component | Composition No. | | | | | | | | |
|---|---|---|---|---|---|---|---|---|---|
| (w/w %) | 28 | 29 | 30 | 31 | 32 | 33 | 34 | 35 | 36 |
| API (%) | 23 | 24 | 25 | 26 | 27 | 28 | 29 | 30 | 31 |
| Excipient 1 | 40-90 | 45-90 | 50-90 | 55-90 | 40-90 | 45-90 | 55-90 | 50-90 | 50-90 |
| Excipient 2 | 5-15 | 1-15 | 5-15 | 1-15 | | | | | 5-15 |
| Excipient 3 | | | | | 1-10 | 1-10 | 1-10 | 1-10 | |
| Excipient 4 | 0-25 | 0-25 | 0-25 | 0-25 | 0-25 | 0-25 | 0-25 | 0-25 | 0-25 |
| Additional | qs | qs | qs | qs | qs | qs | qs | qs | qs |
| Excipients | | | | | | | | | |

The API in this example in specific compositions is TU. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid, octadecanoic acid, or a combination thereof. Excipient 3 in specific compositions is H—(O—CH₂—CH₂)ₙ—OH where n is an integer from 3 to 900 (e.g., PEG having an average molecular weight in the range of 2000-12000). Excipient 4 in specific compositions is (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene. These compositions can be filled into soft gel or hard gel capsules depending on its flowability at the temperatures useful for making these dosage forms.

Shown below are various compositions suitable for oral administration as described herein. In these Examples the amount of excipient adds up to 100% (does not include the API) and the API weight percent is the final weight percent in the pharmaceutical composition.

TABLE E

| Component | Composition No. | | | | | | | | |
|---|---|---|---|---|---|---|---|---|---|
| (w/w %) | 37 | 38 | 39 | 40 | 41 | 42 | 43 | 44 | 45 |
| API | 23 | 24 | 25 | 26 | 27 | 28 | 29 | 30 | 31 |
| Excipient 1 | 45-80 | 45-80 | 45-80 | 45-80 | 45-80 | 45-80 | 45-80 | 45-80 | 45-80 |
| (e.g., Fatty | | | | | | | | | |
| acid) | | | | | | | | | |
| Excipient 2 | 1-15 | 1-15 | 1-15 | 1-15 | 1-15 | 1-15 | 1-15 | 1-15 | 1-15 |
| Excipient 3 | 0-10 | 0-10 | 0-10 | 0-10 | 0-10 | 0-10 | 0-10 | 0-10 | 0-10 |
| Excipient 4 | 0.1-0.3 | 0.1-0.3 | 0.1-0.3 | 0.1-0.3 | 0.1-0.3 | 0.1-0.3 | 0.1-0.3 | 0.1-0.3 | 0.1-0.3 |
| Additional | qs | qs | qs | qs | qs | qs | qs | qs | qs |
| Excipients | | | | | | | | | |

The API in this example in specific compositions is TU. Excipient 1 in specific compositions is (9Z)-octadec-9-enoic acid, hexadecanoic acid or a combination thereof. Excipient 2 in specific compositions is a combination of mono-, di-, or tri-propane-1,2,3-triol esters of octadecanoic acid and hexadecanoic acid. Excipient 3 in specific compositions polyoxylated hydrogenated castor oil (Cremophor R40). Excipient 4 in specific compositions is ascorbyl palmitate. These compositions can be filled into soft gel or hard gel capsules depending on its flowability at the temperatures useful for making these dosage forms.

The API in this example in specific compositions is TU.

These compositions can be made by any suitable method and filled into hard gel or soft gel capsules as appropriate. For example, the one or more of the ingredients are warmed or heated to a temperature that allows for dissolving any solid ingredients, the API is added and mixed until a homogenous mixture is obtained and the capsule can be filled at an appropriate temperature and if needed, allowed to cool to room temperature.

The API in the examples below in specific compositions is TU.

### Composition (100)

| | Quantity Fill Material | |
|---|---|---|
| | per Hard Shell Capsule | |
| Ingredient Name | % w/w | mg |
| API | 23%-28% | 140-275 |
| Oleic Acid, NF | 60%-70% | 450-530 |
| Polyoxyl 40 Hydrogenated Castor Oil, NF | 0%-7% | 0-37 |
| Ascorbyl Palmitate, NF | 0.1%-0.3% | 0.5-2.5 |
| Polyethylene Glycol 8000, NF | 3%-9% | 35-55 |
| Total | 100.0 | Adjustable |

### Composition (101)

| | Quantity Fill | | Quantity Fill |
|---|---|---|---|
| | Material per Hard | | Material per |
| | Gel Capsule | | Soft gel Capsule |
| Ingredient Name | % w/w | mg | mg |
| API | 23%-28% | 140-275 | 140-275 |
| Oleic Acid, NF | 37%-46% | 280-330 | 532-627 |
| Peppermint Oil, NF | 15-21 | 120-150 | 228-285 |
| Polyoxyl 40 Hydrogenated | 0-7% | 0-35 | 0-67 |
| Castor Oil, NF | | | |
| Ascorbyl Palmitate, NF | 0.20 | 0.5-2.5 | 1.0-4.8 |
| Glyceryl Palmitostearate | 9%-15% | 80-100 | 152-190 |
| (Glyceryl Distearate, NF) | | | |
| Total | 100.0 | Adjustable | Adjustable |

### Composition (102)

| | Quantity Fill | | Quantity Fill |
|---|---|---|---|
| | Material per Hard | | Material Soft |
| | Gel Capsule | | gel Capsule |
| Ingredient Name | % w/w | mg | mg |
| API | 25%-32% | 140-275 | 140-275 |
| Oleic Acid, NF | 50%-60% | 340-400 | 646-760 |
| Polyoxyl 40 Hydrogenated | 0%-7% | 21-32 | 0-61 |
| Castor Oil, NF | | | |
| Stearic Acid, NF | 0%-7% | 0-32 | 0-61 |
| Glyceryl Palmitostearate | 3%-13% | 47-58 | 89-110 |
| (Glyceryl Distearate, | | | |
| NF; Precirol ATO 5) | | | |
| Ascorbyl Palmitate, NF | 0.1%-3% | 0.5-2.5 | 1.0-4.8 |
| Total | 100.00 | adjustable | adjustable |

### Composition (103)

| | Quantity Fill Material | |
|---|---|---|
| | per Hard Gel Capsule | |
| Ingredient Name | % w/w | mg |
| API | 27%-33% | 140-275 |
| Oleic Acid, NF | 50%-70% | 335-395 |
| Polyoxyl 40 Hydrogenated Castor Oil, NF | 0%-7% | 0-30 |
| Ascorbyl Palmitate, NF | 0.1%-0.3% | 0.5-2.5 |
| Polyethylene Glycol 8000, NF | 3%-9% | 32-42 |
| Total | 100.0 | adjustable |

These compositions can be made by any suitable method and filled into hard gel or soft gel capsules as appropriate. For example, the one or more of the ingredients are warmed or heated to a temperature that allows for dissolving any solid ingredients, the API is added and mixed until a homogenous mixture is obtained and the capsule can be filled at an appropriate temperature and if needed, allowed to cool to room temperature.
The API in the examples below in specific compositions is TU.

### Composition (104)

| | Weight Percent of Fill |
|---|---|
| | Pharmaceutical |
| | Composition (±1%) |
| Ingredient Name | % w/w |
| API | 24 |
| Oleic Acid, NF | 65 |
| Polyoxyl 40 Hydrogenated Castor Oil, NF | 4 |
| Ascorbyl Palmitate, NF | 0.2 |
| Polyethylene Glycol 8000, NF | 6 |
| Total | 100 |

### Composition (105)

| | Weight Percent of |
|---|---|
| | Fill Pharmaceutical |
| | Composition (±1%) |
| Ingredient Name | % w/w |
| API | 24 |
| Oleic Acid, NF | 41 |
| Peppermint Oil, NF | 18 |
| Polyoxyl 40 Hydrogenated Castor Oil, NF | 4 |
| Ascorbyl Palmitate, NF | 0.2 |
| Glyceryl Palmitostearate (Glyceryl Distearate, NF) | 12 |
| Total | 100 |

### Composition (106)

| | Weight Percent of |
|---|---|
| | Fill Pharmaceutical |
| | Composition (±1%) |
| Ingredient Name | % w/w |
| API | 28 |
| Oleic Acid, NF | 55 |
| Polyoxyl 40 Hydrogenated Castor Oil, NF | 4 |
| Stearic Acid, NF | 4 |
| Glyceryl Palmitostearate (Glyceryl Distearate, | 8 |
| NF; Precirol ATO 5) | |
| Ascorbyl Palmitate, NF | 0.2 |
| Total | 100 |

### Composition (107)

| | Weight Percent of |
|---|---|
| | Fill Pharmaceutical |
| | Composition (±1%) |
| Ingredient Name | % w/w |
| API | 30 |
| Oleic Acid, NF | 59 |
| Polyoxyl 40 Hydrogenated Castor Oil, NF | 4 |
| Ascorbyl Palmitate, NF | 0.2 |
| Polyethylene Glycol 8000, NF | 6 |
| Total | 100 |

### Composition (108)

| | Theoretical Qty. |
|---|---|
| | per Capsule |
| Ingredient Name | % w/w |
| Testosterone undecanoate | 23-35 |
| Oleic Acid, NF | 40-70 |
| Polyoxyl 40 Hydrogenated Castor Oil, NF | 0-10 |
| Stearic Acid, NF | 0-10 |
| Glyceryl Palmitostearate (Glyceryl Distearate, | 1-15 |
| NF; Precirol ATO 5) | |
| Ascorbyl Palmitate, NF | 0.1-3 |
| Total | 100.00 |

As is apparent to the skilled artisan, these formulations can be varied and still yield the beneficial properties described herein. In a particular example, of these compositions and those described throughout this specification are formulations having from 70-80 mg (e.g., 75 mg), 140-160 mg (e.g., 150 mg), 107.5-117.5 mg (e.g., 112.5 mg), 215 to 235 mg (e.g., 225 mg), or 290 to 310 mg (e.g., 300 mg) of API which are particularly preferred and similar in composition (e.g., carriers and amounts) to Compositions (100)-(108). Preferred compositions are those that are pharmaceutically equivalent, bioequivalent or both to those described herein.

One specific formulation having TU as API is as follows.

### Composition (110)

| | Weight Percent of |
|---|---|
| | Fill Pharmaceutical |
| | Composition (±1%) |
| Ingredient Name | % w/w |
| API | 15 |
| Glyceryl Monolinoleate, NF | 63 |
| Polyoxyl 40 Hydrogenated Castor Oil, NF | 15 |
| Ascorbyl Palmitate, NF | 0.2 |
| Polyethylene Glycol 8000, NF | 6 |
| Total | 100 |

Similar composition to Compositions (100)-(108) and (110) can also have for example:
(a) a different fatty acid, an additional fatty acid or both,
(b) a different hydrophilic surfactant, an additional hydrophilic surfactant or both,
(c) a mono- or di-glyceride in place of the fatty acid or in combination with the fatty acid,
(d) a different solidifying agent, an additional solidifying agent, or both,
(e) a different diglyceride than glyceryl palmitostearate, an additional diglyceride or both,
(f) a different antioxidant, an additional antioxidant or both,
(g) have additional additives,
(h) use menthol or another alcohol in place of or in addition to peppermint oil,
(i) use a tocopherol in place of fatty acid, in combination with fatty acid, in place of peppermint oil, in addition to peppermint oil or a combination thereof,
(j) use a different monoglyceride than glyceryl monolinoleate, an additional monoglyceride, a diglyceride in place of glyceryl monolinoleate, a diglyceride in combination with glyceryl monolinoleate or a combination thereof, or
(k) a combination of any of the above.
   Additionally, the percent loading of API can vary in compositions (100)-(108) or (110) or similar compositions as in (a)-(k) or Tables A-E e.g., the API loading can be 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%. It certain aspects, the amount of excipients (e.g., as in (a)-(k) or those of compositions (100)-(108) or (110)) can vary or be selected from those described in Tables A-E.) In some aspects, the composition is bioequivalent to one or more of compositions (100)-(108) or (110) below e.g., when compositions (100)-(108) or (110) have about 75 mg TU, 112.5 mg TU, 150 mg TU, 225 mg TU or 300 mg TU.

Thus, the compositions described herein can also be as follows. According to this embodiment, the dose of TU can be administered as a pharmaceutical composition suitable for oral administration. In one aspect, the pharmaceutical composition is formulated as a tablet or a capsule. In another aspect, the pharmaceutical composition is formulated as a soft gel or hard gel capsule. In one aspect, the pharmaceutical composition comprises from about 10% to 50% testosterone undecanoate. In another aspect, the pharmaceutical composition comprises from about 14% to about 40% testosterone undecanoate. In yet another aspect, the pharmaceutical composition comprises from about 14%-18% testosterone undecanoate. In yet another aspect, the pharmaceutical composition comprises from about 14%-32% testosterone undecanoate. In yet another aspect, the pharmaceutical composition comprises from about 14%-16% testosterone undecanoate. In yet another aspect, the pharmaceutical composition comprises from about 24%-34% testosterone undecanoate. In yet another aspect, the pharmaceutical composition comprises from about 26%-33% testosterone undecanoate. In one aspect, the pharmaceutical composition comprises an amount of TU ranging from about 70 mg to about 320 mg, loading of TU as described in this paragraph, and a lipophilic additive. In one aspect, the pharmaceutical composition further comprises a hydrophilic additive or surfactant. In one aspect, the lipophilic additive comprises a lipophilic surfactant. In one aspect, the pharmaceutical composition comprises a mono or diglyceride where the fatty acid moiety has from 8-20 carbons and 0, 1, 2 or 3 unsaturations. In one aspect, the pharmaceutical composition comprises an 8-20 carbon fatty acid having 0, 1, 2 or 3 unsaturations. In one aspect, the pharmaceutical composition comprises a solidifying agent. In one aspect, the pharmaceutical composition is flowable at a temperature below 40° C. In one aspect, the pharmaceutical composition is flowable at a temperature sufficient for the production of soft gel capsules (e.g., commercial quantities). In one aspect, the pharmaceutical composition is flowable at a temperature sufficient for the production of hard gel capsules (e.g., commercial quantities). In one aspect, the solidifying agent is a polyethylene glycol. In some aspects, the solidifying agent is stearic acid, glyceryl palmitostearate or a combination thereof. In some aspects, the composition is bioequivalent or pharmaceutically equivalent to any specific composition described herein when administered in a similar fashion under appropriate conditions.

It is understood that the above-described various types of compositions, dosage forms and/or modes of applications are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, but not limited to, variations in size, materials, shape, form, function and manner of operation, assembly and use may be made without departing from the principles and concepts set forth herein.

### Release Profile of Unit Dosage Form Containing Testosterone Undecanoate

In another embodiment, the unit dosage form has a release profile (e.g., single or multiple point) of TU using a USP type 2 apparatus in about 1000 mL 8% Triton X100 solution in water at a specific temperature (e.g., 20.0, 37.0 or 40.0° C. (±0.5)) at 100 rpm that releases at least 10, 20, 30, 40, 50, 60, 70, 75, 80, 85, 90, 95, 96, 97, 98, or 99% at 15, 20, 30, 40, 45, 50, 60, 90, 120, 180, 240, or 300 minutes. In a specific aspect, the unit dosage form having or made from solid releases greater than 85% at 4 hours; greater than 70% at 2 hours; or greater than 60% at 1 hour. In a specific aspect, the unit dosage form has a release profile that releases less than 100% (or 95%) at 15 minutes or less than 100% (or 95%) at 30 minutes. In a specific aspect, the unit dosage form releases less than 10, 20, 30, 40, 50, 60, 70, 75, 80, 85, 90, 95, 96, 97, 98, or 99% at 15, 20, 30, 40, 45, 50, 60, 90, 120, 180, 240, or 300 minutes.

### Methods of Preparing Unit Dosage Forms

In one specific aspect, the carrier(s) and API are brought to or maintained at a temperature at which they are flowable (e.g., above 10° C., 20° C., 25° C., 30° C., 35° C., or 40° C.). In one aspect, the mixture of carrier and API is a clear solution at a specified temperature (e.g., above 10° C., 20° C., 25° C., 30° C., 35° C., or 40° C.). In one aspect, the mixture of carrier and API is a cloudy or hazy solution at a specified temperature (e.g., below 10° C., 20° C., 25° C., 30° C., 35° C., or 40° C.).

In one example, the composition is prepared by weighing all of the components, except the API into a clean stainless steel container and mixed together at ambient temperature or at elevated temperatures e.g., at about 25° C. to about 30° C., at about 30° C. to about 35° C., at about 35° C. to about 40° C., at about 40° C. to about 45° C., at about 45° C. to about 45° C., or 50° C. to about 70° C., using a stirrer. The API is added and stirred into the mixture of other components until the API dissolves. A predetermined quantity of this “liquid fill material” is disposed into a capsule (for example, hard gelatin capsule) to get the required API dose per dosage unit. The capsules are allowed to cool at room temperature, banded (if required) and packaged in a HDPE bottle and tightly closed with an appropriate lid. It is noted that various capsule sizes (e.g., hard gel or soft gel) are available to the skilled artisan and allow for variations in the amount of loading of API in mg per unit dosage form. Typically, soft gel capsules for oral administration have fill volumes of less than 1.5 mL, 1.3 mL or 1.25 mL with numerous incremental fill volumes in these ranges. Similarly, hard gel capsules typically have fill volumes of less than 1.25 mL, 1.10 mL or 1 mL. Due to the nature of some hard gel capsules, the total fill volume may not be useable. There is a practical limit on the temperature at which capsules can be filled—for example temperature above 40° C. typically melt, deform, or otherwise damage soft gel capsules typically employed in the industry. Hard gel capsules are typically less sensitive to temperature and can be filled at higher temperatures e.g., above 40° C.

In certain embodiments, any pharmaceutical composition described herein, e.g., a can be prepared by (I) combining and heating all ingredients until a molten mixture is obtained (e.g., 50-70° C.); and (ii) encapsulating an amount of molten mixture comprising a select dose (e.g., a therapeutically effective amount or a partial dose of a therapeutically effective amount) API to obtain an oral dosage form. In certain instances, the molten mixture is spray-congealed to obtain beads. In some instances, the molten mixture is sprayed onto inert cores (e.g., sugar spheres) to obtain coated cores. In certain embodiments, such beads, cores, or similar forms are encapsulated or otherwise formulated to provide an oral dosage form. In some instances, the molten mixture is admixed, uniformly dispersed, or granulated over a carrier and compressed into a tablet dosage form. In certain embodiments, prior to compression, the molten mixture/carrier composition is further mixed with one or more pharmaceutical aid including, by way of non-limiting example, glidants, lubricants, binders, or the like. In some embodiments, the carrier is a therapeutically inert carrier such as, by way of non-limiting example, microcrystalline cellulose, starch, lactose, or the like.

In various embodiments, pharmaceutical compositions described herein are formulated as oral dosage forms. Oral dosage forms are prepared by any suitable process including one or more steps of, by way of non-limiting example, agglomeration, air suspension chilling, air suspension drying, balling, coacervation, comminution, compression, pelletization, cryopelletization, encapsulation, extrusion, granulation, homogenization, inclusion complexation, lyophilization, nanoencapsulation, melting, mixing, molding, pan coating, solvent dehydration, sonication, spheronization, spray chilling, spray congealing, spray drying, or the like.

In some embodiments, a pharmaceutical composition described herein is formulated with a substrate to form an oral dosage form. In various embodiments, substrates useful for formulating pharmaceutical compositions described herein as oral dosage forms include or comprise, by way of non-limiting example, a powder or a multiparticulate (e.g., one or more granules, one or more pellets, one or more beads, one or more spherules, one or more beadlest, one or more microcapsules, one or more millispheres, one or more mini capsules, one or more microcapsules, one or more nanocapsules, one or more nanospheres, one or more microspheres, one or more minitablets, one or more tablets, one or more capsules, or one or more combinations thereof). In certain instances, a powder constitutes a finely divided (milled, micronized, nanosized, precipitated) form of an active ingredient or additive molecular aggregates or a compound aggregate of multiple components or a physical mixture of aggregates of an active ingredient and/or additives.

The following examples are provided to promote a more clear understanding of certain embodiments of this disclosure and are in no way meant as a limitation thereon.

### Example

A clinical trial was performed as set forth below in regards to the TRT disclosed herein.

### Key Inclusion Criteria:

Diagnosis of primary or secondary hypogonadism
Male, 18 to 80 years of age, hypogonadism onset prior to age 65
Serum T<300 ng/dL, based on 2 consecutive samples, obtained on two separate days (6 to 10 AM) either naïve or following appropriate washout.

### Key Exclusion Criteria

IPSS≧19 points
PSA>2 ng/mL

### Body Mass Index≧38 Kg/m2

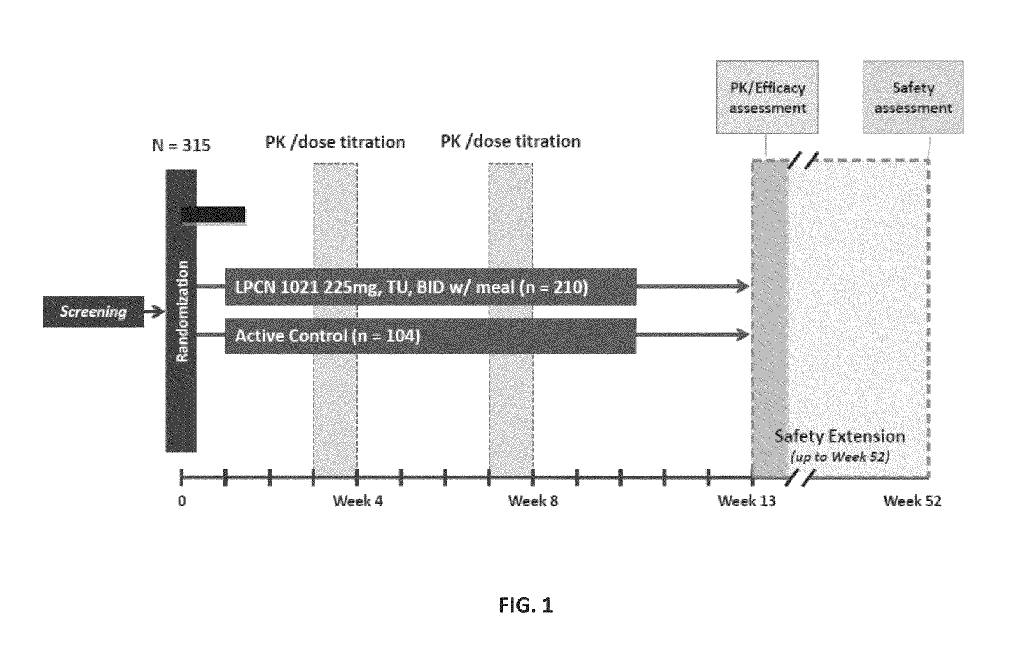

Hemoglobin <11.5 or >16.5 g/dL; Hematocrit <35 or >54%
History of myocardial infarction; prostate or breast cancer; uncontrolled, untreated obstructive sleep apnea; gastric surgery, HIV, Hepatitis, seizures or convulsions
Subjects with a partner who is pregnant or planning to become pregnant during the study
A total of 314 subjects were enrolled and randomized into the TRT arm or active control arm.
The clinical trial design is outlined in FIG. 1.
Starting dose: 225 mg testosterone undecanoate (TU) (equivalent to 142 mg testosterone) twice daily, 12 hours apart, with standard meals
Two dose titration periods based on full 24 hour PK profile at Week 3 and 7

### Titration Metric:

If C_{avg(0-24)}<300 ng/dL, up titrate by 75 mg TU/dose up to a maximum dose of 300 mg
If C_{avg(0-24)}>1140 ng/dL, down titrate by 75 mg TU/dose to a minimum dose of 150 mg
If Cₘₐₓ>1500 ng/dL, down titrate by 75 mg TU/dose to a minimum dose of 150 mg, irrespective of C_{avg(0-24)} value.
The dose range was 150 mg to 300 mg BID (total daily dose of 300 mg to 600 mg TU).

### Primary Analysis Set

Efficacy Population (N=153): Subjects randomized into the study with at least one PK profile and no significant protocol deviations (includes imputed missing data/LOCF)

### Other Analysis Sets

Safety Set (N=210): Subjects randomized into the study and took at least one dose of the drug
Imputed missing data by Last Observation Carried Forward (LOCF), or considered treatment failures if no PK data available
Full Analysis Set (N=193): Subjects randomized into the study with at least one PK profile (includes imputed missing data/LOCF)
Per Protocol Set (N=132): Subjects who completed Week 13 without significant protocol deviations
Primary endpoint target ≧75% subjects should achieve Cavg within normal range (300 ng/dL to 1140 ng/dL) and ≧65% lower bound 95% CI

### % of Subjects with C_{avg} in Various Ranges

| | Efficacy | Per Protocol | Full Analysis | Safety |
|---|---|---|---|---|
| Measure | Population | Set | Set | Set |
| Number of subjects | 152* | 132 | 192* | 210 |
| % subjects with Cavg | 88.2% | 87.9% | 87.5% | 80.0% |
| within normal range | | | | |
| 95% CI lower bound | 81.9% | 81.1% | 82.0% | 73.8% |

### % of Subjects with C_{avg} in Various Ranges

| Range | Efficacy Population |
|---|---|
| C_{avg} < 300 ng/dL | 11.2% |
| C_{avg} between 300 and 1140 ng/dL | 88.2% |
| C_{avg} > 1140 ng/dL | 0.7% |
| Parameter | Mean (CV) |
| C_{avg} (ng/dL) | 447 (37%) |

### Proportion of Subjects Achieving Maximum Serum Total T Concentrations (Cₘₐₓ) in Predefined Cₘₐₓ Range

| Measure | FDA Threshold | Efficacy Population |
|---|---|---|
| Number of subjects | 152* | |
| Cₘₐₓ < 1500 ng/dL | ≧85% | 82.9% |
| 1800 ≦ Cₘₐₓ ≦ 2500 ng/dL | ≦5% | 4.6% |
| Cₘₐₓ > 2500 ng/dL | None | 2.0% |

### Final Dose Distribution of Subjects in Efficacy Population

| Dose (mg TU, BID) | % of subjects |
|---|---|
| 150 | 35% |
| 225 | 52% |
| 300 | 13% |

### Effective Dose and Titration Regimen in Efficacy Population

| | % of |
|---|---|
| Parameter | subjects |
| % subjects requiring no dose change | 41% |
| % subjects requiring one dose change | 44% |
| (either after week 3 or week 7) | |
| % subjects requiring two dose changes | 15% |
| (both after week 3 and 7) | |

### Comparison of TRT Disclosed Herein to Rextoro®

The initial starting daily dose for Rextoro® was 632 mg TU. The initial starting dose for the TRT disclosed herein was 450 mg TU.

### Comparison to Rextoro®

| Parameters | TU TRT Disclosed Herein | Rextoro ® |
|---|---|---|
| Efficacy | Unequivocally efficacious | Marginal efficacy |
| | and acceptable sensitivity | in their pivotal |
| | analysis (88.2%) | study (75.0%) |
| Mean C_{avg} (ng/dL) | 447 ng/dL | 422 ng/dL |
| Subjects with | 11% | 23% |
| C_{avg} < 300 ng/dL | | |
| Variability (CV) | C_{avg}: 37%; Cₘₐₓ: 45% | C_{avg}: 41%; Cₘₐₓ: 55% |
| Starting dose | 52% of subjects ended | 23% of subjects ended |
| | up on starting dose | up on starting dose |
| Titration visit | 85% of subjects require no | Unknown |
| | more than one titration visit | |
| AM-PM effect | Not much different AM- | Different AM-PM |
| | PM effect | effect (Increased |
| | | absorption at PM) |

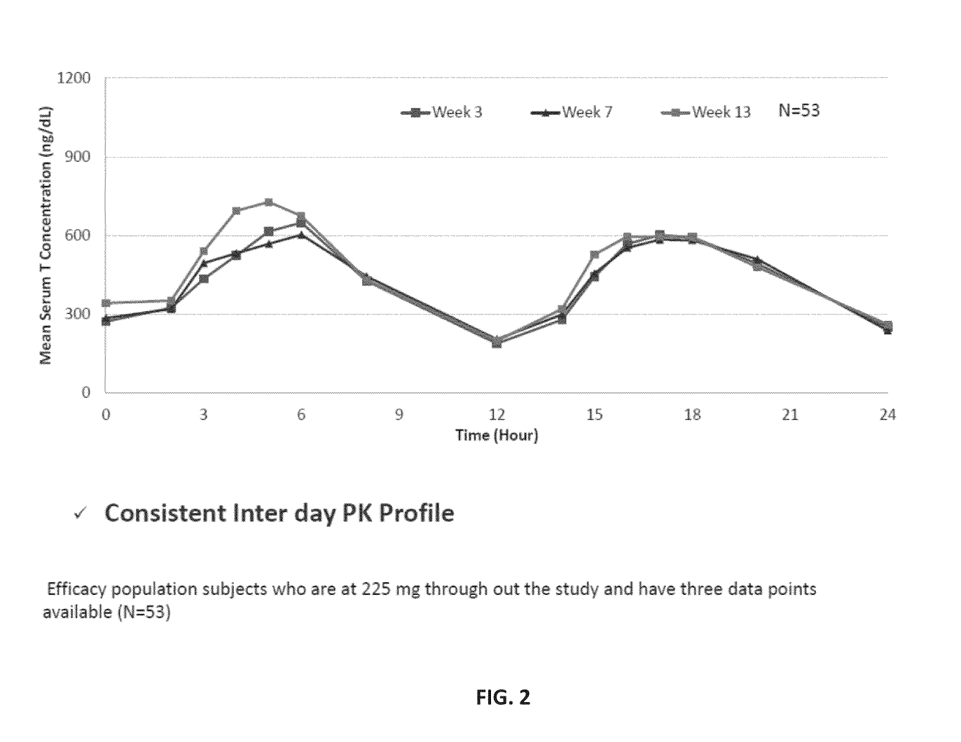

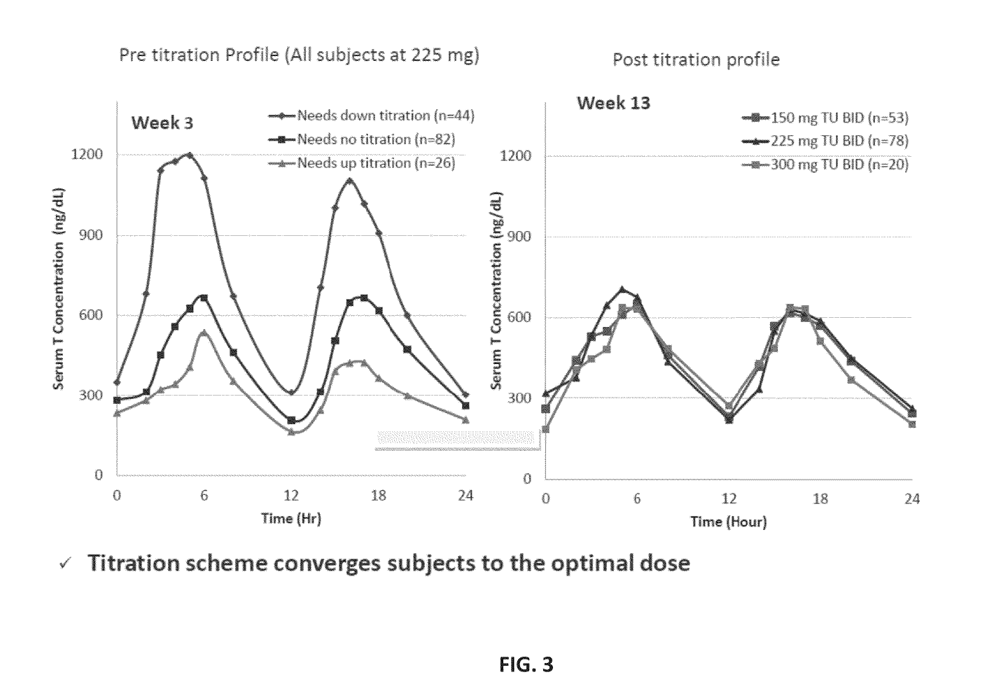

The results described in this example and by extension to the various embodiments described herein illustrate the unique efficacy and safety characteristics of the testosterone replacement therapy provided herein. The Tables in this Example illustrate the robustness of the testosterone replacement therapy in comparison to a product (Rextoro®) that had undergone two phase III trials yet failed to be approved by the United States FDA. As can be seen from the data, the testosterone replacement therapy has unexpectedly properties compared to Rextoro® including efficacy and robustness/simplicity of dosing regimens. For example, the TRT disclosed herein compared to Rextoro® had excellent efficacy (and acceptable sensitivity analysis), lower number of dose changes, a higher Cave, lower starting dose etc. FIG. 2 shows the 24 hour pharmacokinetic profiles at weeks 3, 7 and 13 of the patients did not undergo a dose change during the clinical trial (and had three PK profiles). It can be seen that the therapy yield consistent inter-day pharmacokinetics. FIG. 3 shows how the 24 hour pharmacokinetic profiles of the patients needing an up titration or a down titration converged to the profile of those patients not needing a titration.

What is claimed is:

1. A unit dosage form comprising:
about 75 mg of testosterone undecanoate, about 112.5 mg of testosterone undecanoate, about 150 mg testosterone undecanoate, about 225 mg testosterone undecanoate or about 300 mg testosterone undecanoate; and
a pharmaceutically acceptable carrier,
wherein said unit dosage form is for oral administration, or a pharmaceutically equivalent version thereof, or a bioequivalent version thereof.

2. The unit dosage form of claim 1 wherein when 1, 2, 3, or 4 unit dosage forms are administered with a meal twice a day to a hypogonadal male therapeutically effective C_{avg} serum testosterone levels are provided.

3. The unit dosage form of claim 1 which is a tablet or capsule.

4. The unit dosage form of claim 1 said pharmaceutically acceptable carrier comprising glyceryl monolinoleate, Cremophor RH 40, oleic acid, polyethylene glycol, stearic acid, glyceryl palmitostearate, ascorbyl palmitate, or a combination thereof.

5. The unit dosage form of claim 1 wherein when tested using a USP type 2 apparatus in about 1000 mL 8% Triton X100 solution in water at 37.0±0.5 at 100 rpm releases at least 60% at 15 minutes and less than 100%.

6. A testosterone replacement therapy for twice daily dosing, said therapy comprising:
(a) 2 different dose strength oral dosage forms having different amounts of testosterone undecanoate;
(b) 3 dosing regimens providing for 3 different daily doses of testosterone undecanoate;
(c) both (a) and (b); or
(d) a pharmaceutically equivalent version thereof, a bioequivalent version thereof or both.

7. The testosterone replacement therapy of claim 6 which provides steady state serum levels of testosterone (C_{avg}) to a male having testosterone deficiency or in need of said therapy in the range of about 300 ng/dL to about 1140 ng/dL.

8. The testosterone replacement therapy of claim 6 which provides steady state serum levels of testosterone (C_{avg}) to a male having testosterone deficiency or in need of said therapy in the range of about 435 ng/dL to about 1140 ng/dL.

9. The testosterone replacement therapy of claim 6 which provides single dose Cₘₐₓ levels of serum testosterone at steady state to a population of males having testosterone deficiency, or in need of said therapy, of less than 2500 ng/dL in at least 95% of the population of males, less than 1500 ng/dL in at least 85% of the population of males; or a serum testosterone Cₘₐₓ of about 1800 ng/dL to about 2500 ng/dL in 10% or less of the population of males having testosterone deficiency.

10. The testosterone replacement therapy of claim 6 wherein one of the dosage forms has from about 140 to 160 mg testosterone undecanoate and the other dosage form has from about 215 mg to about 235 mg testosterone undecanoate.

11. The testosterone replacement therapy of claim 6 wherein one of the of dosage forms has from about 145 to 155 mg testosterone undecanoate and the other dosage form has from about 220 mg to about 230 mg testosterone undecanoate.

12. The testosterone replacement therapy of claim 6 wherein one the of dosage forms has about 150 mg testosterone undecanoate and the other dosage form has about 225 mg testosterone undecanoate.

13. The testosterone replacement therapy of claim 6 wherein one of the dosage forms has from about 60 to 90 mg testosterone undecanoate and the other dosage form has from about 100 mg to about 130 mg testosterone undecanoate.

14. The testosterone replacement therapy of claim 6 wherein one of the dosage forms has from about 70 to 80 mg testosterone undecanoate and the other dosage form has from about 107 mg to about 118 mg testosterone undecanoate.

15. The testosterone replacement therapy of claim 6 wherein one of the dosage forms has about 75 mg testosterone undecanoate and the other dosage form has about 112.5 mg testosterone.

16. The testosterone replacement therapy of claim 6 wherein 3 dosing regimens providing for 3 different daily doses of testosterone undecanoate provides for a first daily dose of about 275 mg to about 325 mg of testosterone undecanoate, a second daily dose of from about 425 mg to about 475 mg of testosterone undecanoate and a third daily dose of about 575 mg to about 625 mg of testosterone undecanoate.

17. The testosterone replacement therapy of claim 6 wherein 3 different daily doses of testosterone undecanoate provides for a first daily dose of about 300 mg of testosterone undecanoate, a second daily dose of about 450 testosterone undecanoate and a third daily dose of about 600 mg of testosterone undecanoate.

18. The testosterone replacement therapy of claim 6 wherein said therapy comprises:
2 dosage forms, one having about 75 mg of testosterone undecanoate and the other dosage form having about 112.5 testosterone undecanoate;
3 dosing regimens, a first dosing regimen comprising administration of two dosage forms twice a day each dosage form having about 75 mg testosterone undecanoate, a second dosing regimen comprising administration of two dosage forms twice a day each dosage form having about 112.5 mg testosterone undecanoate, and a third dosing regimen comprising administration of four dosage forms twice a day each dosage form having about 75 mg testosterone undecanoate; or a pharmaceutically equivalent version thereof.

19. The testosterone replacement therapy of claim 6 wherein said therapy comprises:
2 dosage forms, one having about 150 mg of testosterone undecanoate and the other dosage form having about 225 testosterone undecanoate;
3 dosing regimens, a first dosing regimen comprising administration of one dosage form twice a day each dosage form having about 225 mg testosterone undecanoate, a second dosing regimen comprising administration of two dosage forms twice a day each dosage form having about 150 mg testosterone undecanoate, and a third dosing regimen comprising administration of one dosage form twice a day said dosage form having about 150 mg testosterone undecanoate; or a pharmaceutically equivalent version thereof.

20. A testosterone replacement therapy for twice daily dosing said therapy comprising: oral administration of a pharmaceutical composition comprising testosterone undecanoate at 3 dose titration levels or a pharmaceutically equivalent version thereof.

21. A therapy for treating a male having a baseline serum testosterone level of 300 ng/dL or less said therapy comprising:
(a) 2 unit dosage forms differing in amount of testosterone undecanoate;
(b) 3 dosing regimens differing in total daily dose of testosterone undecanoate;
(c) both (a) and (b); or
(d) a pharmaceutically equivalent version thereof, a bioequivalent version thereof, or both.

22. The testosterone replacement therapy of claim 6 comprising an initial dosing regimen and a maintenance dosing regimen.

23. The testosterone replacement therapy of claim 6 comprising a dose titration determination.

24. The testosterone replacement therapy of claim 6 comprising an initial dosing regimen which provides a daily dose of about 450 mg of testosterone undecanoate.

25. The testosterone replacement therapy of claim 6 comprising an initial dosing regimen which provides a daily dose of about 450 mg of testosterone undecanoate and a maintenance regimen daily dose of about 600 mg of testosterone undecanoate.

26. The testosterone replacement therapy of claim 6 comprising an initial dosing regimen which provides a daily dose of about 450 mg of testosterone undecanoate and a maintenance regimen daily dose of about 300 mg of testosterone undecanoate.

27. The testosterone replacement therapy of claim 6 wherein a dose titration determination is made by measuring serum testosterone levels within a specified window of time after single dose administration of testosterone undecanoate said specified window of time having serum testosterone levels that correlate to C_{avg} values of 1140 ng/dL or greater; 300 ng/dL or lower; or between 300 ng/dL and 1140 ng/dL.

28. The testosterone replacement therapy of claim 6 wherein a dose titration is made by measuring serum testosterone levels within a specified window of time after single dose administration of testosterone undecanoate said specified window of time correlating to serum testosterone C_{avg} values of 1140 ng/dL or greater; 300 ng/dL or lower; or between 300 ng/dL and 1140 ng/dL and when serum testosterone levels within the specified window of time correlate to a serum testosterone C_{avg} of: 1140 ng/dL or more, a dose reduction is made; 300 ng/dL or lower, a dose increase is made; or between 300 ng/dL and 1140 ng/dL no dose adjustment is made.

29. The testosterone replacement therapy of claim 6 wherein a patient receiving the therapy has a dose titration assessment.

30. The testosterone replacement therapy of claim 29 wherein the dose titration assessment comprises determining a value of serum testosterone at from about two to eight hours after receiving a single dose of testosterone undecanoate at steady state.

31. The testosterone replacement therapy of claim 30 wherein a patient having (a) a low serum testosterone level at two to eight hours after receiving a dose of testosterone undecanoate receives a higher dose of testosterone undecanoate; (b) a high serum testosterone level at two to eight hours after receiving a dose of testosterone undecanoate receives a lower dose of testosterone undecanoate; (c) an intermediate serum testosterone level receives an intermediate dose of testosterone undecanoate; or (d) a combination thereof.

32. The testosterone replacement therapy of claim 29 wherein the dose titration assessment comprises determining the serum testosterone level during a window period within two to eight hours after receiving a dose of testosterone undecanoate.

33. The testosterone replacement therapy of claim 32 wherein the window period is the titration correlation window for a representative population.

34. The testosterone replacement therapy of claim 6 wherein the testosterone undecanoate is administered with a meal.

35. The testosterone replacement therapy of claim 6 comprising a dose titration or adjustment wherein a subject using the therapy has a serum testosterone level 3-6 hours after single dose administration with a meal at steady state is (a) less than about 125 ng/dL, the dose of TU is adjusted; (b) greater than about 1200 ng/dL, the dose of TU is adjusted down; or (c) between about 125 ng/dL and 1200 ng/dL, the dose of TU remains the same.

36. The testosterone replacement therapy of claim 6 comprising a dose titration or adjustment wherein a subject using the therapy has a serum testosterone level 3-6 hours after single dose administration with a meal at steady state is (a) less than about 125 ng/dL, the dose of TU is adjusted up an additional 75 mg TU per dose; (b) greater than about 1200 ng/dL, the dose of TU is adjusted down by 75 mg TU per dose or (c) between about 125 ng/dL and 1200 ng/dL, the dose of TU remains the same.
